SEQUENCE LISTING (6 sequences)

SEQ ID NO: 1 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 2 - protein, 640 aa, Homo sapiens
AKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 3 - DNA, 2445 bp, Homo sapiens
ataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccggcatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgattggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattaggggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccattttttaagttggttacttcaaagtaaataaactttaaaattcaaaaaaaaaaaaaaaaaa

SEQ ID NO: 4 - protein, 641 aa, Homo sapiens; identical to SEQ ID NO: 1

SEQ ID NO: 5 - protein, 640 aa, Homo sapiens; identical to SEQ ID NO: 2

SEQ ID NO: 6 - DNA, 2551 bp, Homo sapiens
ggaaaacggccagcctgaggagctgctgcgagggtccgcttcgtctttcgagagtgactcccgcggtcccaaggctttccagagcgaacctgtgcggctgcaggcaccggcgtgttgagtttccggcgttccgaaggactgagctcttgtcgcggatcccgtccgccgtttccagcccccagtctcagagcggagcccacagagcagggcaccggcatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgatcggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggttctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctggcggcttcggggctcagggtcccaagggagggtctgggtcaggccctaccattgaggaggtggattaggggcctttgttctttagtatgtttgtctttgaggtggactgttgggactcaaggactttgctgctgttttcctatgtcatttctgcttcagctctttgctgcttcacttctttgtaaagttgtaacctgatggtaattagctggcttcattatttttgtagtacaaccgatatgttcattagaattctttgcatttaatgttgatactgtaagggtgtttcgttccctttaaatgaatcaacactgccaccttctgtacgagtttgtttgttttttttttttttttttttttttgcttggcgaaaacactacaaaggctgggaatgtatgtttttataatttgtttatttaaatatgaaaaataaaatgttaaactttaaaaaaaaaaaaaaaaaaaaaaaaaaaaaaa

(19) United States
(12) Patent Application Publication
(10) Pub. No.: US 2020/0085812 A1
(43) Pub. Date: Mar. 19, 2020

(54) HEAT SHOCK PROTEIN INDUCERS AND FRONTOTEMPORAL DISORDERS

(71) Applicants: Orphazyme A/S, Copenhagen N (DK); UCL Business PLC, London (GB)

(72) Inventors: Thomas Kirkegaard Jensen, Rødovre (DK); Linda Greensmith, London (GB)

(21) Appl. No.: 16/615,893

(22) PCT Filed: May 24, 2018
(86) PCT No.: PCT/EP2018/063662

§ 371 Date: Nov. 22, 2019

(30) Foreign Application Priority Data
May 24, 2017 (EP) EP17172669.8

(51) Int. Cl. A61K 31/4545; A61K 38/17; A61K 38/47; A61P 25/00

(52) U.S. Cl. CPC A61K 31/4545; A61P 25/00; A61K 38/47; A61K 38/17

(57) ABSTRACT

The present invention relates to a bioactive agent that increases the intracellular concentration and/or activity of one or more heat shock proteins, including Hsp70, for use in the treatment of frontotemporal disorders.

### TECHNICAL FIELD

The present invention relates to a bioactive agent that increases the intracellular concentration and/or activity of one or more heat shock proteins, including Hsp70, for use in the treatment of frontotemporal disorders such as frontotemporal dementia.

### BACKGROUND

Heat shock proteins are found in all compartments of a cell where conformational rearrangements of proteins occur. Heat shock proteins are also commonly known as molecular chaperones, as they serve to keep their client proteins in a proper, folded state. Protein synthesis is the major source of unfolded peptides in the cell but a challenge to the cell by high temperature or other stressful stimuli that render proteins structurally labile and hence prone to unfolding and aggregation is met with a specific cellular response involving the increased production of Heat shock proteins. This response is a phenomenon observed in every cell type ranging from prokaryotes to eukaryotes and is referred to as the heat-shock- or stress-response. The proteins induced by this response are known as the heat shock proteins (HSPs), of which there exist several families.

A primary example of a family of HSPs is the Hsp70 proteins. This family has recently been implicated in other aspects of cellular homeostasis besides serving as a molecular chaperone—most markedly through its anti-apoptotic features, its functions in immunity, and the apparent dependence of cancer cells on the upregulation of Hsp70. Furthermore, Hsp70 can serve a role in safeguarding lysosomal integrity.

HSP gene expression and protein expression can be amplified by HSP inducers. Examples of small molecule inducers of the heat shock response, including Hsp70, include bimoclomol, arimoclomol, iroxanadine and BGP-15.

The term frontotemporal disorder refers to changes in behavior and thinking that are caused by underlying brain diseases collectively called frontotemporal lobar degeneration (FTLD). FTLD is not a single brain disease but rather a family of neurodegenerative diseases, any one of which can cause a frontotemporal disorder. Frontotemporal dementia (FTD) on the other hand is one of several possible variations and is sometimes more precisely called behavioral variant frontotemporal dementia, or bvFTD.

Dementia results in severe loss of thinking abilities that interferes with a person's ability to perform daily activities. An estimated 10% of all cases of dementia are caused by FTLD and may be as common as Alzheimer's among people younger than age 65.

A main histological subtype of FTLD is FTLD-TDP (or FTLD-U) characterized by ubiquitin and TDP-43 positive, tau negative, FUS (fused in sarcoma/translocated in sarcoma) negative inclusions.

Mutations in valosin-containing protein (VCP) cause a multisystem disorder that includes inclusion body myopathy (IBM) associated with Paget's disease of the bone (PDB) and fronto-temporal dementia (FTD); or IBMPFD. Although IBMPFD is a multisystem disorder, muscle weakness is the presenting symptom in greater than half of patients and an isolated symptom in 30%. Patients with the full spectrum of the disease make up an estimated 12% of those affected; therefore it is important to consider and recognize IBMPFD in a neuromuscular clinic. In addition to myopathic features; vacuolar changes and tubulofilamentous inclusions are found in a subset of patients. The most consistent findings are VCP, ubiquitin and TAR DNA-binding protein 43 (TDP-43) positive inclusions.

Mutations in the VCP gene are also reported to be the cause of 1%-2% of familial amyotrophic lateral sclerosis (fALS) cases, potentially causing sporadic ALS-FTD.

RNA granules are microscopically visible cellular structures that aggregate by protein-protein and protein-RNA interactions. RNA granule formation relies on the multivalency of RNA and multi-domain proteins as well as low-affinity interactions between proteins with prion-like/low-complexity domains (e.g. FUS and TDP-43). Classes of these structures include nucleoli, Cajal bodies, nuclear speckles and paraspeckles in the nucleus, as well as P-bodies and stress granules in the cytoplasm.

Unlike other RNA granules, cytoplasmic stress granules are not constitutively present; instead, their formation is induced by cellular stress, such as heat shock or oxidative stress, and they disassemble once the perturbation subsides. Notably, morphologically similar cytoplasmic inclusions are observed in neurons of patients with amyotrophic lateral sclerosis (ALS), frontotemporal lobar degeneration (FTLD) and other age-related neurodegenerative disease, which often exhibit compositional overlap with endogenous stress granules (15,16).

Symptoms of frontotemporal dementia progress at a rapid, steady rate. There are currently no treatments available to prevent, stop or reverse frontotemporal dementia.

WO 2009/155936 discloses Hsp70 and inducers thereof for treating lysosomal storage diseases. WO 2005/041965 discloses use of the heat shock protein inducer arimoclomol for treating neurodegenerative diseases, including ALS.

### SUMMARY

The present inventors now find that mutant VCP (mVCP) mice not only show degenerative muscle pathology but also CNS pathology with motoneuron (motor neuron) loss in the spinal cord (ALS phenotype) and abnormal TDP-43, ubiquitin, p-tau, p62 and LC3 in the brain (FTD phenotype). As shown herein, all these features are seen to be attenuated in mVCP mice treated with an inducer of the heat shock proteins, including Hsp70 and co-chaperones. Furthermore, it is shown herein that mVCP mouse brains display stress granule protein markers and that treatment with an inducer of the heat shock proteins, including Hsp70 and co-chaperones, attenuate the appearance of said stress granule protein markers.

It is thus an aspect to provide a bioactive agent that increases the intracellular concentration and/or activity of one or more heat shock proteins, including Hsp70, for use in the treatment of frontotemporal disorders.

In one embodiment said bioactive agent increases the intracellular concentration and/or activity of Hsp70, i.e. is an inducer of Hsp70, such as a small molecule inducer of Hsp70, such as an inducer selected from the group consisting of arimoclomol, iroxanadine, bimoclomol, BGP-15, their stereoisomers and the acid addition salts thereof.

In one embodiment the frontotemporal disorder is selected from the group consisting of frontotemporal lobar degeneration (FTLD), frontotemporal dementia (FTD), inclusion body myopathy (IBM) with FTD, Paget's disease of bone (PDB) with FTD, IBM with early-onset PDB and FTD (IBMPFD), FTD with amyotrophic lateral sclerosis (ALS) (ALS-FTD), and IBM with FTD, PDB and ALS (IBMPFD-ALS).

### DESCRIPTION OF DRAWINGS

FIG. 1. A-C) Typical traces of functional motor units recruited in the EDL muscle of wtVCP, mVCP and Arimoclomol treated mVCP mice. D) The Bar chart shows the mean percentage of motor units in each group. WT=non-transgenic controls. E) The Bar chart shows maximal tetanic force generated by the EDL muscle in anaesthetised mice. n=10 animals per group; *=P<0.05.

FIG. 2. A-C) Images of spinal cord cross-sections stained for Nissl (gallacyanin), with sciatic pool motor neurons circled. D) The Bar chart shows the mean motor neuron survival (% of WT) in mice of each experimental group. WT=non-transgenic controls (n=5 animals per group); *=P<0.0001. E) The histogram shows motor neuron size distribution from the sciatic pool of the spinal cord of mice of each group.

FIG. 3. Sections of the ventral horn of the spinal cord showing the sciatic pool of WT non-transgenic mice, mutant VCP (mVCP) mice and mVCP mice treated with Arimoclomol, immunostained for TDP-43 (Green). Top panels: TDP-43 immuno-reactivity in spinal cord sections shows only nuclear labelling in WT controls, nuclear and cytoplasmic labelling in the mVCP spinal cords, and reduced cytoplasmic labelling in spinal cords of mVCP mice treated with Arimoclomol. Bottom panel: corresponding images showing co-staining for TDP-43 immuno-reactivity (green) and DAPI labelling of the nuclei (blue).

FIG. 4. A) Sections of the cortical region of brains taken from WT non-transgenic mice, mutant VCP mice and mVCP mice treated with Arimoclomol. Top panels: sections immunostained for TDP-43 (Green). Bottom panels: sections co-stained for TDP-43 immunoreactivity (green) and the nuclear label DAPI (blue). Scale bar=20 μm. TDP-43 immunoreactivity shows only nuclear labelling in brains of WT mice, nuclear and cytoplasmic labelling in sections from mVCP mice and reduced cytoplasmic staining in sections of mVCP mice treated with Arimoclomol. B) Higher magnification images of TDP-43 immunoreactivity in sections of brain from mVCP mice show abnormal nuclear clearance of TDP-43. C) Sections of the cortical region of brains from WT non-transgenic mice, mutant VCP mice and mVCP mice treated with Arimoclomol, immunostained for ubiquitin (top panel; red) and p-Tau (bottom panel; red). The white arrow indicates phosphorylated-tau in a large extracellular lesion in the cortex of a mVCP mouse. DAPI labels nuclei (blue)/Scale bar=10 μm. Top panel: Ubiquitin immunoreactivity in brain sections shows no positive ubiquitin labelling in sections from WT control mice, cytoplasmic ubiquitin-positive aggregates in sections of mVCP mice, but almost no ubiquitin staining in sections of mVCP mice treated with Arimoclomol. DAPI labels the nuclei. Bottom panel: Beta-Ill Tubulin labels neuronal cells. In WT brain sections p-tau immunostaining is observed within the cells, mainly in the nucleus. In mVCP mice, p-tau positive lesions surrounded by neuronal cells were detected. In Arimoclomol treated mVCP mice, P-tau staining was similar to that of WT controls.

FIG. 5: Immunostaining of spinal cord sections from wt-VCP control mice, untreated mVCP and Arimoclomol treated mVCP mice. A) Immunostaining for Ubiquitin (green) and co-staining for the nuclear marker DAPI (blue) reveals cytoplasmic aggregates in neurons in sections from mVCP mice but not in wtVCP neurons or mVCP mice treated with Arimoclomol. B) Immunostaining of spinal cord sections for p62 and co-staining for myelin (red) reveals i) little if any p62 staining in sections from wt-VCP mice; ii) increased expression of p62 in mVCP spinal cord, co-localised with myelin (red); iii) a similar pattern of p62 expression in spinal cord of arimoclomol treated mVCP mice to that observed in control mice. C) (i) Immunostaining of spinal cord of mVCP mice for p62 shows a specific increase in p62 expression in white matter and p62-positive aggregates in motor neurons (magnified inset). ii) Higher magnification image of spinal cord white matter shows of mVCP mice shows disrupted myelin structure and increased p62 expression. D) LC3 immunostaining in spinal cord sections from i) wt-VCP mice, ii) mVCP mice and iii) arimoclomol-treated mVCP mice shows an increase in LC3 expression in white matter of mVCP spinal cord which was not observed in wt VCP or arimoclomol treated mVCP mice.

FIG. 6: Sections of the cortical region of brains from WT non-transgenic mice, mutant VCP mice and mVCP mice treated with arimoclomol. Top Panel: sections were immunostained for ubiquitin (red) and co-stained with the nuclear marker DAPI (blue). There was no ubiquitin immunoreactivity in brain sections of wt-VCP mice, but cytoplasmic ubiquitin-positive aggregates were observed in sections from mVCP mice; there was little if any ubiquitin staining in sections of mVCP mice treated with Arimoclomol. Middle Panel: sections were immunostained for phosphorylated tau (p-Tau; red) and co-stained for the neuronal marker Beta-Ill Tubulin (green). In sections from wt-VCP control mice, p-tau immunostaining was observed within neurons, mainly located in the nucleus. In mVCP mice, p-tau positive lesions surrounded by neuronal cells were detected; a phosphorylated-tau positive large extracellular lesion in the cortex of a mVCP mouse is indicated by the white arrow. In arimoclomol treated mVCP mice, the pattern of p-tau staining was similar to that of WT controls. Bottom panel: Sections of the cortical region of mVCP mice stained for phosphorylated tau (red), and co-stained for either (i) the neuronal marker β-III tubulin (green), (ii) the microglial marker Iba1 (green) or iii) the astroglial marker GFAP (red; p-tau=green), and co-stained with the nuclear marker DAPI (blue). (i) Extracellular lesions positive for p-tau were only observed in mVCP mouse brain, surrounded by β-III tubulin-positive neurons; (ii) p-tau aggregates were associated with Iba1-positive microglia and (iii) GFAP-positive glial cells. Scale bar=10 μm.

FIG. 7. A) Sections of the cortical region of brains of WT non-transgenic (wtVCP) mice, mutant VCP mice and mVCP mice treated with Arimoclomol, immunostained for Hsp70 (green), the neuronal marker β-III tubulin (red) and the nuclear marker DAPI (blue). Scale bar=10 μm. Top panel: wtVCP mice show little HSP70 expression in the brain. Middle panel: mVCP mice show an increase in HSP70 expression. Bottom panel: HSP70 expression is enhanced in mVCP mice treated with Arimoclomol. B) Immunostaining of sections of arimoclomol treated mVCP mice show that HSP70 expression (green) is augmented in beta-Ill-negative (glial) cells. Scale bar=10 μm

FIG. 8. Immunostaining of spinal cord sections from wt-VCP control mice, untreated mVCP and Arimoclomol treated mVCP mice. A) Sections were stained for HSP70 (green) and the neuronal marker β-III tubulin (red), co-stained with the nuclear marker DAPI (blue). Hsp70 expression was very low in the spinal cord of wtVCP mice, increased in mutant VCP, and further enhanced in spinal cords of Arimoclomol-treated mVCP mice, mainly in non-neuronal cells, glial cells (white arrows). Scale bar=10 μm. B) The Bar chart shows the quantification of fluorescence intensity of HSP70 immunoreactivity in the spinal cord of mice from each group, and confirms the increased expression of HSP70 in mVCP mice is enhanced in mVCP mice treated with arimoclomol. C) Immunostaining of spinal cords of mVCP mice for HSP70 (green) and the astroglial marker GFAP (red) shows that HSP70 expression is increased in mVCP glial cells (yellow arrows) labelled in the adjacent section with GFAP (red). White arrows indicate neuronal cells also positive for HSP70.

FIG. 9. Brain sections of wt-VCP, mVCP and arimoclomol treated mVCP mice stained with Sudan Black. A) A low magnification image of a Sudan Black stained brain section is shown for reference, indicating the area of motor cortex shown in higher magnification in images in B). B) Sections were stained with the TUNEL assay (green) to detect apoptotic cells, and co-stained with the nuclear marker DAPI. Apoptotic cells were detected in sections treated with nuclease which acted as a positive control as well as in sections of cortex from mVCP mice (green, white arrow). This assay only labels nuclei of cells undergoing programmed cell death. Inset images show corresponding DAPI-labelled nuclei.

FIG. 10. Mouse brains were immunostained for the stress granule markers Tia1, FMRP and G3BP (green, white arrows), and co-stained for the nuclear marker DAPI (inserts; blue). Stress granule markers were aggregated in sections from mVCP mouse brain. No staining for stress granules was observed in the brains of control mice or in mVCP mice treated with Arimoclomol. Scale bar=20 μm

FIG. 11. The pattern of innervation of the neuromuscular junction (NMJ) of soleus muscles of wt-VCP, mVCP and mVCP mice treated with arimoclomol was examined by immunostaining for presynaptic markers—neurofilaments (NF; green) or the synaptic vesical protein SV2 (green) and co-labelling with α-bungarotoxin (α-Btx; red) which labels postsynaptic acetylcholine receptors. A) A NMJ in the soleus muscle of a wt-VCP mouse, showing a typical innervated endplate. B) Images of NMJs from mVCP mice showing i) a denervated NMJ, with no contact between the axon (green) and endplate (red); ii) and iii) disrupted endplates in mVCP NMJs. C) NMJs in soleus muscles from Arimoclomol-treated mVCP mice i) and ii) showing innervated NMJs, with co-labelling between pre- and postsynaptic markers (yellow staining). Scale bar=10 μm

FIG. 12. Human induced pluripotent stem cell (iPSC) derived motor neurons differentiated from mutant VCP patients and healthy controls were immunostained for TDP-43 and co-stained with the nuclear marker DAPI. A) TDP-43 immunoreactivity (green) shows normal nuclear localisation of the TDP-43 in iPSC-derived motor neurons in cells derived from healthy controls, cytoplasmic mislocalisation of TDP-43 in iPSC-derived motor neurons from mVCP patients, with nuclear clearance of TDP-43 in some cells. In contrast, in iPSC-derived motor neurons from mVCP patient cells treated with Arimoclomol, TDP-43 expression was largely nuclear, with a similar pattern of expression to that observed in healthy controls. B) Immunostaining for HSP70 (green), plus and minus the neuronal marker β-III tubulin (red), and co-stained for the nuclear marker DAPI. HSP70 was expressed at low levels in control cells, but was increased in motor neurons derived from mVCP patients; HSP70 was further enhanced in cells from mVCP patients cells treated with Arimoclomol. DAPI labels nuclei (blue). Scale bar=20 μm.

FIG. 13. Sections of post-mortem human brain cortex from patients with Frontotemporal Dementia (FTD) associated with either motor neuron disease (FTD-MND), with ubiquitin-positive inclusions (FTD-U), with mutant TDP-43 (FTD-TDPA), or with tau-positive inclusions (FTD-tau), compared to samples of the same region of brain from healthy controls. A) Sections were immunostained for TDP-43 (green) and co-stained with the nuclear marker DAPI (blue). Cytoplasmic mislocalisation of TDP-43 was observed in all patient samples, while this was only rarely observed in control tissue. B) Sections were immunostained for HSP70 expression (green) and co-stained with the nuclear marker DAPI (blue). HSP70 expressing was increased in all patient samples compared to healthy controls. Scale bar=10 μm

FIG. 14. Sections of post-mortem human brain cortex from patients with Frontotemporal Dementia (FTD) associated with either motor neuron disease (FTD-MND), with ubiquitin-positive inclusions (FTD-U), with mutant TDP-43 (FTD-TDPA), or with tau-positive inclusions (FTD-tau), compared to samples of the same region of brain from healthy controls. The sections were immunostained for the autophagy markers LC3 and p62. Cytoplasmic aggregates of both LC3 and p62 were observed in all FTD patient tissues assessed (black arrows and inset). p62 expression was also seen in some neurites in FTD-U and FTD-MAPT patient brains and intensely labelled neurites were observed in FTD-TDPA (white arrows). In sections from patients with FTD-MAPT, p62 was seen to associate with neurofibrillary tangles. Scale bar=10 μm

### DETAILED DESCRIPTION

The present inventors have identified TDP-43 mislocalisation, ubiquitin aggregation, p-tau lesions, p62 and LC3 expression and stress granule formation in mutant VCP mice as well as port-mortem human brain cortex from patients with Frontotemporal Dementia (FTD).

The effect of inducing the heat shock response, including the effect on heat shock proteins, such as Hsp70 and co-chaperones, observed herewith on abnormal TDP-43, ubiquitin, p-tau, p62, LC3 and stress granule markers in the brain has potential in therapies involving frontotemporal disorders and FTD-like pathologies associated with one or more of TDP-43 mislocalisation, ubiquitin aggregation, p-tau lesions, p62 and LC3 expression and stress granule formation; such as for example TDP-43 mislocalisation, ubiquitin aggregation, p-tau lesions, p62 and LC3 expression and stress granule formation caused by a VCP mutation.

It is thus an aspect of the present disclosure to provide a bioactive agent as defined herein that increases the intracellular concentration (or levels) and/or activity of one or more heat shock proteins, including Hsp70, for use in the treatment of a frontotemporal disorder.

In one embodiment said frontotemporal disorder is associated with frontotemporal dementia.

In one embodiment there is provided use of a bioactive agent as defined herein that increases the intracellular concentration and/or activity of one or more heat shock proteins, including Hsp70, for the manufacture of a medicament for the treatment of a frontotemporal disorder.

In one embodiment there is provided a method of treating a frontotemporal disorder, said method comprising one or more steps of administering a bioactive agent as defined herein that increases the intracellular concentration and/or activity of one or more heat shock proteins, including Hsp70, to an individual in need thereof.

The term “Individual” or “subject” refers to vertebrates, in particular a member of a mammalian species, preferably primates including humans. In a preferred embodiment, an individual as used herein is a human being, male or female, of any age.

An “individual in need thereof” refers to an individual who may benefit from the present treatment. In one embodiment, said individual in need thereof is a diseased individual, wherein said disease is associated with one or more of TDP-43 mislocalisation, ubiquitin aggregation p-tau lesions, p62 and LC3 expression or aggregation and stress granule formation, and/or associated with a VCP mutation, such as frontotemporal disorders as defined herein.

In one embodiment, said treatment may be prophylactic, curative or ameliorating. In one particular embodiment, said treatment is prophylactic. In another embodiment, said treatment is curative. In a further embodiment, said treatment is ameliorating.

The bioactive agents that increase the intracellular concentration and/or activity of one or more heat shock proteins, including Hsp70, are defined in detail herein below, and encompass inducers of heat shock proteins including Hsp70.

The diseases associated with TDP-43 mislocalisation, ubiquitin aggregation, p-tau lesions, p62 and LC3 expression (or aggregation) and/or stress granule formation and/or a VCP mutation are defined in detail herein below, and encompass frontotemporal lobar degeneration (FTLD) or FTLD-TDP, frontotemporal dementia (FTD) including FTD-MND, FTD-U, FTD-TDPA and FTD-tau, inclusion body myopathy (IBM) with FTD, Paget's disease of bone (PDB) with FTD, IBM with early-onset PDB and FTD (IBMPFD), FTD with amyotrophic lateral sclerosis (ALS) (ALS-FTD), and IBM with FTD, PDB and ALS (IBMPFD-ALS).

Frontotemporal Disorders

Frontotemporal disorders are the result of damage to neurons in the frontal and temporal lobes of the brain. Frontotemporal disorders refer to changes in behaviour and thinking that are caused by underlying brain diseases collectively called frontotemporal lobar degeneration (FTLD). FTLD is not a single brain disease but rather a family of neurodegenerative diseases, any one of which can cause a frontotemporal disorder. FTLD encompasses the subgroups frontotemporal dementia (FTD), progressive nonfluent aphasia (PFNA), and semantic dementia (SD).

A main histological subtype of FTLD is FTLD-TDP (or FTLD-U) characterized by ubiquitin and TDP-43 positive, tau negative, FUS (fused in sarcoma/translocated in sarcoma) negative inclusions.

Frontotemporal disorders thus comprise frontotemporal lobar degeneration (FTLD), FTLD-TDP, frontotemporal dementia (FTD) including FTD-MND, FTD-U, FTD-TDPA and FTD-tau, inclusion body myopathy (IBM) with FTD, Paget's disease of bone (PDB) with FTD, IBM with early-onset PDB and FTD (IBMPFD), FTD with amyotrophic lateral sclerosis (ALS) (ALS-FTD), and IBM with FTD, PDB and ALS (IBMPFD-ALS).

In one embodiment of the present disclosure there is provided a bioactive agent as defined herein for use in the treatment of a frontotemporal disorder. In one embodiment the frontotemporal disorder is selected from the group consisting of frontotemporal lobar degeneration (FTLD) and FTLD-TDP, frontotemporal dementia (FTD), inclusion body myopathy (IBM) with FTD, Paget's disease of bone (PDB) with FTD, IBM with early-onset PDB and FTD (IBMPFD), FTD with amyotrophic lateral sclerosis (ALS) (ALS-FTD), and IBM with FTD, PDB and ALS (IBMPFD-ALS).

In one embodiment of the present disclosure there is provided a bioactive agent as defined herein for use in the treatment of a frontotemporal disorder selected from the group consisting of frontotemporal lobar degeneration (FTLD) and FTLD-TDP, frontotemporal dementia (FTD), inclusion body myopathy (IBM) with FTD, Paget's disease of bone (PDB) with FTD, IBM with early-onset PDB and FTD (IBMPFD), FTD with amyotrophic lateral sclerosis (ALS) (ALS-FTD), and IBM with FTD, PDB and ALS (IBMPFD-ALS).

In one embodiment said frontotemporal disorder or frontotemporal lobar degeneration (FTLD) is associated with (or displays or show symptoms of) frontotemporal dementia (FTD).

In one embodiment the frontotemporal dementia (FTD) is selected from the group consisting of frontotemporal Dementia (FTD) associated with motor neuron disease (FTD-MND), frontotemporal Dementia (FTD) associated with ubiquitin-positive inclusions (FTD-U), frontotemporal Dementia (FTD) associated with mutant TDP-43 (FTD-TDPA) and frontotemporal Dementia (FTD) associated with tau-positive inclusions (FTD-tau).

In one embodiment said frontotemporal disorder is associated with a mutation in the VCP gene (mVCP), or displays a mutation in the VCP gene (mVCP). In one embodiment said frontotemporal disorder comprising frontotemporal lobar degeneration (FTLD), FTLD-TDP, frontotemporal dementia (FTD) including FTD-MND, FTD-U, FTD-TDPA and FTD-tau, inclusion body myopathy (IBM) with FTD, Paget's disease of bone (PDB) with FTD, IBM with early-onset PDB and FTD (IBMPFD), FTD with amyotrophic lateral sclerosis (ALS) (ALS-FTD), and IBM with FTD, PDB and ALS (IBMPFD-ALS) is associated with a mutation in the VCP gene (mVCP), or displays a mutation in the VCP gene (mVCP).

“Associated with a mutation in the VCP gene” in the present context means that the patient presenting with the given disease is identified as having a mutation in the VCP gene.

Hence in one embodiment of the present disclosure there is provided a bioactive agent as defined herein for use in the treatment of a frontotemporal disorder, wherein said patient having a frontotemporal disorder has a mutation in the VCP gene (mVCP).

In one embodiment said frontotemporal disorder is associated with a mutation in the VCP gene causing TDP-43 mislocalisation and/or ubiquitin aggregation and/or p-tau lesions, and/or p62 and LC3 expression and/or stress granule formation. In one embodiment said frontotemporal disorder is associated with TDP-43 mislocalisation and/or ubiquitin aggregation and/or p-tau lesions, and/or p62 and LC3 expression or aggregation and/or stress granule formation.

In one embodiment said frontotemporal disorder comprising frontotemporal lobar degeneration (FTLD), FTLD-TDP, frontotemporal dementia (FTD) including FTD-MND, FTD-U, FTD-TDPA and FTD-tau, inclusion body myopathy (IBM) with FTD, Paget's disease of bone (PDB) with FTD, IBM with early-onset PDB and FTD (IBMPFD), FTD with amyotrophic lateral sclerosis (ALS) (ALS-FTD), and IBM with FTD, PDB and ALS (IBMPFD-ALS) is associated with TDP-43 mislocalisation and/or ubiquitin aggregation and/or p-tau lesions, and/or p62 and LC3 expression or aggregation and/or stress granule formation.

“Associated with TDP-43 mislocalisation and/or ubiquitin aggregation and/or p-tau lesions, and/or p62 and LC3 expression and/or stress granule formation” in the present context means that the patient presenting with the given disease is identified as having TDP-43 mislocalisation and/or ubiquitin aggregation and/or p-tau lesions, and/or p62 and LC3 expression and/or stress granule formation; such as TDP-43 cytoplasmic mislocalisation and/or cytoplasmic ubiquitin aggregation and/or p-tau lesion formation, and/or p62 expression or cytoplasmic aggregation and/or LC3 expression or cytoplasmic aggregation and/or stress granule formation.

In one embodiment said frontotemporal disorder is associated with stress granule formation. In one embodiment said frontotemporal disorder is associated with stress granule formation including one or more of the stress granule markers Tia1, FMRP (Fragile X Mental Retardation protein) and G3BP (RasGAP SH3 domain Binding Protein).

In one embodiment of the present disclosure there is provided a bioactive agent as defined herein for use in the treatment of a frontotemporal disorder selected from the group consisting of frontotemporal lobar degeneration (FTLD), frontotemporal dementia (FTD), inclusion body myopathy (IBM) with FTD, Paget's disease of bone (PDB) with FTD, IBM with early-onset PDB and FTD (IBMPFD), FTD with amyotrophic lateral sclerosis (ALS) (ALS-FTD), and IBM with FTD, PDB and ALS (IBMPFD-ALS); wherein said frontotemporal disorder is associated with a mutation in the VCP gene, and/or wherein said frontotemporal disorder is associated with one or more of TDP-43 mislocalisation, ubiquitin aggregation, p-tau lesions, p62 or LC3 expression (or aggregation) or stress granule formation.

VCP (Uniprot-P55072 (TERA_HUMAN)), or Transitional endoplasmic reticulum ATPase (TER ATPase), is an enzyme that in humans is encoded by the VCP gene. The main function of VCP is to segregate protein molecules from large cellular structures such as protein assemblies, organelle membranes and chromatin, and thus facilitate the degradation of released polypeptides by the multi-subunit protease proteasome. VCP gene codes for the protein VCP, which is a member of the AAA-ATPase (ATPases associated with diverse cellular activities) superfamily, and is involved in cell cycle control, membrane fusion, and the ubiquitin-proteasome degradation pathway.

In one embodiment of the present disclosure, the frontotemporal disorder as defined herein is associated with a mutation of the VCP gene selected from the group consisting of R93C, R95G, R95C, R95H, I126F, P137L, R155S, R155C, R155H, R155P, R155L, G157R, R159C, R159H, R159G, R191Q, L198W, A232E, T262A, N387H, A439P, A439S and D592N.

In one embodiment of the present disclosure there is provided a bioactive agent as defined herein that increases the intracellular concentration and/or activity of one or more heat shock proteins, including Hsp70, for use in the treatment of frontotemporal lobar degeneration (FTLD).

Frontotemporal dementia (FTD) is a term for a diverse group of uncommon disorders that primarily affect the frontal and temporal lobes of the brain. It is characterized by progressive neuronal loss and typical loss of over 70% of spindle neurons, while other neuron types remain intact. Although FTDs are clinically, genetically and neuropathologically heterogeneous, more than 95% of cases are TDP-43 proteinopathies or taupathies. FTD was originally called “Pick's disease”, a term now reserved for Pick disease, one specific type of FTD.

Some people with FDT undergo dramatic changes in their personality and become socially inappropriate, impulsive or emotionally indifferent, while others lose the ability to use language. Currently, there is no cure for FTD; only treatments that help alleviate symptoms are available.

Subtypes of FTD are identified clinically according to the symptoms that appear first and most prominently. Clinical diagnoses include behavioral variant FTD (bvFTD), primary progressive aphasia (PPA) which affects language, and the movement disorders progressive supranuclear palsy (PSP) and corticobasal degeneration (CBD).

In one embodiment of the present disclosure there is provided a bioactive agent as defined herein that increases the intracellular concentration and/or activity of one or more heat shock proteins, including Hsp70, for use in the treatment of frontotemporal dementia (FTD).

In one embodiment of the present disclosure there is provided a bioactive agent as defined herein that increases the intracellular concentration and/or activity of one or more heat shock proteins, including Hsp70, for use in the treatment of frontotemporal dementia (FTD) selected from the group consisting of frontotemporal Dementia (FTD) associated with motor neuron disease (FTD-MND), frontotemporal Dementia (FTD) associated with ubiquitin-positive inclusions (FTD-U), frontotemporal Dementia (FTD) associated with mutant TDP-43 (FTD-TDPA) and frontotemporal Dementia (FTD) associated with tau-positive inclusions (FTD-tau).

The symptoms and pathology of FTD vary depending on the specific mutation. The majority of FTD patients with a genetic cause have a mutation occurring in one of the following genes: C9orf72; Microtubule-associated protein tau (MAPT, often referred to as “tau”); Progranulin (GRN or PGRN) and Valosin-Containing Protein (VCP). Three additional genes that have been associated with very rare FTD cases: Charged multivesicular body protein 2B (CHMP2B), TAR DNA-binding protein (TARDBP) and Fused in sarcoma (FUS).

In one embodiment, the frontotemporal disorder is Pick disease (PiD).

In one embodiment of the present disclosure there is provided a bioactive agent as defined herein that increases the intracellular concentration and/or activity of one or more heat shock proteins, including Hsp70, for use in the treatment of frontotemporal dementia (FTD) associated with a mutation in the VCP gene.

In another embodiment, the frontotemporal disorder is IBM with early-onset PDB and FTD (IBMPFD) (also termed IBM associated with PDB and FTD).

In one embodiment of the present disclosure there is provided a bioactive agent as defined herein that increases the intracellular concentration and/or activity of one or more heat shock proteins, including Hsp70, for use in the treatment of IBM with early-onset PDB and FTD (IBMPFD).

IBMPFD is a multisystem degenerative disorder that is characterized by inclusion body myopathy (IBM) which results in muscle weakness that sets in during adulthood, early-onset Paget's disease of bone (PDB), and premature FTD. It spreads to other systems and results in respiratory or cardiac failure.

PDB is caused by the excessive breakdown and formation of bone, followed by disorganized bone remodeling. This causes bones to grow larger and weaker than normal, resulting in pain, misshapen bones, fractures and arthritis in the joints near the affected bones. PDB can co-occur with FTD.

In one embodiment, the frontotemporal disorder is inclusion body myopathy (IBM) with FTD (IBM-FTD).

In one embodiment, the frontotemporal disorder is Paget's disease of bone (PDB) with FTD (PDB-FTD).

IBMPFD is a rare disorder in which affected individuals may have muscle weakness, Paget's disease of bone and/or dementia. Muscle weakness in this disorder has typically been attributed to a disease of muscle known as inclusion body myopathy (IBM). The major genetic cause of IBMPFD is mutation of the VCP (valosin-containing protein) gene. Mutations in VCP have also been reported to cause familial ALS (amyotrophic lateral sclerosis) and ALS sometimes occurs in families with IBMPFD. Thus, a condition comprising both IBMPFD and ALS is also identified and may be denoted IBMPFD-ALS (IBM with FTD, PDB and ALS). This condition has also been called multisystem proteinopathy (MSP).

In one embodiment, the frontotemporal disorder is IBMPFD-ALS.

In one embodiment of the present disclosure there is provided a bioactive agent as defined herein that increases the intracellular concentration and/or activity of one or more heat shock proteins, including Hsp70, for use in the treatment of IBMPFD-ALS.

Amyotrophic lateral sclerosis (ALS) has mainly been described as a neurological disorder that affects the motor system, but is now recognized as a multisystem neurodegenerative disease due to the fact that other than motor areas of the brain undergo degeneration. Both FTD and ALS are heterogeneous at the clinical, neuropathological and genetic levels and, even though they come across as distinct progressive disorders, there is increasing evidence of the fact that they share some clinical, neuropathological and genetic features.

ALS can co-occur with any of the FTLD clinical variants, but is most commonly associated with FTD (otherwise known as behavioral variant FTD or bvFTD).

In one embodiment, the frontotemporal disorder is FTD with amyotrophic lateral sclerosis (ALS) (ALS-FTD).

In one embodiment, the frontotemporal disorder is bvFTD with amyotrophic lateral sclerosis (ALS) (ALS-bvFTD).

In one embodiment of the present disclosure there is provided a bioactive agent as defined herein that increases the intracellular concentration and/or activity of one or more heat shock proteins, including Hsp70, for use in the treatment of ALS-FTD.

In one embodiment, the frontotemporal disorder is fALS associated with mVCP (VCP-fALS).

In one embodiment, the frontotemporal disorder is sporadic ALS-FTD.

Bioactive Agent

A “Bioactive agent” (i.e., biologically active substance/agent) is any agent, drug, substance, compound, composition of matter or mixture which provides some pharmacologic, often beneficial, effect that can be demonstrated in vivo or in vitro. As used herein, this term further includes any physiologically or pharmacologically active substance that produces a localized or systemic effect in an individual. Further examples of bioactive agents include, but are not limited to, agents comprising or consisting of an oligosaccharide, a polysaccharide, an optionally glycosylated peptide, an optionally glycosylated polypeptide, a nucleic acid, an oligonucleotide, a polynucleotide, a lipid, a fatty acid, a fatty acid ester and secondary metabolites.

A bioactive agent as defined herein increases the intracellular concentration (or levels) and/or activity of one or more heat shock proteins, in one embodiment including Hsp70 and co-chaperones. In one embodiment said bioactive agent is selected from:
Inducers of heat shock proteins, including Hsp70, such as Hsp70 inducers
   small molecule inducers of heat shock proteins, including Hsp70;
      hydroxylamine derivatives, e.g. bimoclomol, arimoclomol, iroxanadine and BGP-15
   Membrane fluidizers, such as benzyl alcohol
   Sub-lethal heat-therapy (≤42° C.) or hyperthermia
   Certain anti-inflammatory and anti-neoplastic drugs
   Cellular stressors;
      Reactive oxygen species (ROS); Adrenalin, noradrenalin; UV light; Radiation therapy,
Hsp70 protein, or a functional fragment or variant thereof.

A bioactive agent as defined herein is thus any agent, chemical or compound that increases the intracellular concentration and/or activity of one or more heat shock proteins, in one embodiment including Hsp70 and co-chaperones; and includes Hsp70 itself, or a functional fragment or variant thereof, any heat shock protein includes and any Hsp70 inducer known to the skilled person.

A bioactive agent that increases the intracellular concentration and/or activity of one or more heat shock proteins, including Hsp70, and a bioactive agent that increases the intracellular concentration and/or activity of Hsp70, can be used interchangeably with ‘Hsp70 inducer’ herein.

An Hsp70 inducer can amplify Hsp70 gene expression and protein expression with or without a concomitant stress. A direct Hsp70 inducer is a compound that can by itself amplify Hsp70 gene expression and protein expression without a concomitant stress. An indirect Hsp70 inducer, or an Hsp70 co-inducer, is a compound that cannot amplify Hsp70 gene expression and protein expression without a concomitant (mild) stress, but the stress-induced increase in Hsp70 levels is further elevated or enhanced by their presence.

It follows that a bioactive agent may increase the intracellular concentration and/or activity of heat shock proteins, such as Hsp70, either directly or indirectly.

In one embodiment, the bioactive agent is Hsp70, or a functional fragment or variant thereof.

In another embodiment, the bioactive agent is an inducer of heat shock proteins, including Hsp70.

In one embodiment the inducer of heat shock proteins, including Hsp70, is an inducer of one or more of Hsp70, Hsp40, Hsp72 and Hsp90, and co-chaperones.

In one embodiment the inducer of heat shock proteins is an inducer of at least Hsp70.

In one embodiment the inducer of heat shock proteins is an inducer of Hsp70.

Reference to an inducer of Hsp70, or inducing Hsp70, implies that at least Hsp70 is induced, and does not exclude co-induction of other proteins and effectors such as other heat shock proteins. An inducer of Hsp70 refers equally to Hsp70 inducers and co-inducers, and direct and indirect Hsp70 inducers.

In one embodiment, the bioactive agent comprises a combination of Hsp70, or a functional fragment or variant thereof, and an inducer of heat shock proteins including Hsp70.

In one embodiment, the bioactive agent reduces cytoplasmic ubiquitin aggregation. In another embodiment, the bioactive agent reduces Transactive response DNA binding protein 43 kDa (TDP-43) cellular mislocalisation. In yet another embodiment, the bioactive agent reduces motor unit loss. In one embodiment, the bioactive agent reduces stress granule formation, such as reduces stress granule markers including Tia1, FMRP and G3BP. In one embodiment, the bioactive agent reduces p-tau positive lesions. In one embodiment, the bioactive agent reduces P62 and/or LC3 expression or cytoplasmic aggregation.

Inducers of Heat Shock Proteins, Including Hsp70

In one embodiment the bioactive agent activates the heat shock response. In one embodiment the bioactive agent increases the intracellular concentration and/or activity of one or more heat shock proteins, including Hsp70. In one embodiment the bioactive agent increases the intracellular concentration (or level) and/or activity of Hsp70. In one embodiment the bioactive agent increases the intracellular concentration (or level) of Hsp70. In one embodiment the bioactive agent is an inducer of one or more heat shock proteins, including Hsp70. In one embodiment the bioactive agent is an inducer of Hsp70.

It is an aspect of the present disclosure to provide an inducer of one or more heat shock proteins, including Hsp70, for use in treating frontotemporal disorders.

In one embodiment there is provided use of an inducer of one or more heat shock proteins, including Hsp70, for the manufacture of a medicament for the treatment of a frontotemporal disorder.

In one embodiment there is provided a method of treating a frontotemporal disorder, said method comprising one or more steps of administering an inducer of one or more heat shock proteins, including Hsp70, to an individual in need thereof.

Small Molecule Inducers of Heat Shock Proteins

In one embodiment the bioactive agent is an inducer of one or more heat shock proteins, including Hsp70. In one embodiment the bioactive agent is a small molecule inducer of heat shock proteins, including Hsp70, such as a small molecule inducer of Hsp70.

In one embodiment an inducer of Hsp70; or a small molecule inducer of one or more heat shock proteins, including Hsp70; is a compound capable of increasing the intracellular concentration (or level) of inter alia Hsp70, such as by amplifying Hsp70 gene expression. An inducer of Hsp70 may also induce other heat shock proteins.

In one embodiment the bioactive agent is capable of increasing the intracellular concentration (or levels) of Hsp70 by amplifying Hsp70 gene expression. In one embodiment the bioactive agent is capable of increasing the intracellular concentration (or level) of Hsp70 by amplifying Hsp70 gene expression, wherein said bioactive agent is a hydroxylamine derivative, such as a hydroxylamine derivative small molecule.

Examples of such hydroxylamine derivatives include arimoclomol, iroxanadine, bimoclomol, BGP-15, their stereoisomers and the acid addition salts thereof.

It is an aspect of the present disclosure to provide a small molecule inducer of one or more heat shock proteins, including Hsp70, for use in treating a frontotemporal disorder.

In one embodiment there is provided use of a small molecule inducer of one or more heat shock proteins, including Hsp70, for the manufacture of a medicament for the treatment of a frontotemporal disorder.

In one embodiment there is provided a method of treating a frontotemporal disorder, said method comprising one or more steps of administering a small molecule inducer of one or more heat shock proteins, including Hsp70, to an individual in need thereof.

Arimoclomol

In one embodiment the small molecule inducer of Hsp70 is selected from N-[2-hydroxy-3-(1-piperidinyl)-propoxy]-pyridine-1-oxide-3-carboximidoyl chloride (arimoclomol), its stereoisomers and the acid addition salts thereof. Arimoclomol is further described in e.g. WO 00/50403.

In one embodiment the small molecule inducer of Hsp70 is selected from N-[2-hydroxy-3-(1-piperidinyl)-propoxy]-pyridine-1-oxide-3-carboximidoyl chloride (arimoclomol), its optically active (+) or (−) enantiomer, a mixture of the enantiomers of any ratio, and the racemic compound, furthermore, the acid addition salts formed from any of the above compounds with mineral or organic acids constitute objects of the present disclosure. All possible geometrical isomer forms of N-[2-hydroxy-3-(1-piperidinyl)-propoxy]-pyridine-1-oxide-3-carboximidoyl chloride belong to the scope of the disclosure. The term “the stereoisomers of N-[2-hydroxy-3-(1-piperidinyl)-propoxy]-pyridine-1-oxide-3-carboximidoyl chloride” refers to all possible optical and geometrical isomers of the compound.

If desired, the N-[2-hydroxy-3-(1-piperidinyl)-propoxy]-pyridine-1-oxide-3-carboximidoyl chloride or one of its optically active enantiomers can be transformed into an acid addition salt with a mineral or organic acid, by known methods.

In one embodiment the small molecule inducer of Hsp70 is the racemate of N-[2-hydroxy-3-(1-piperidinyl)-propoxy]-pyridine-1-oxide-3-carboximidoyl chloride.

In one embodiment the small molecule inducer of Hsp70 is an optically active stereoisomer of N-[2-hydroxy-3-(1-piperidinyl)-propoxy]-pyridine-1-oxide-3-carboximidoyl chloride.

In one embodiment the small molecule inducer of Hsp70 is an enantiomer of N-[2-hydroxy-3-(1-piperidinyl)-propoxy]-pyridine-1-oxide-3-carboximidoyl chloride.

In one embodiment the small molecule inducer of Hsp70 is selected from the group consisting of (+)-R—N-[2-hydroxy-3-(1-piperidinyl)-propoxy]-pyridine-1-oxide-3-carboximidoyl chloride and (−)-(S)—N-[2-hydroxy-3-(1-piperidinyl)-propoxy]-pyridine-1-oxide-3-carboximidoyl chloride.

In one embodiment the small molecule inducer of Hsp70 is an acid addition salt of N-[2-hydroxy-3-(1-piperidinyl)-propoxy]-pyridine-1-oxide-3-carboximidoyl chloride.

In one embodiment the small molecule inducer of Hsp70 is selected from the group consisting of N-[2-hydroxy-3-(1-piperidinyl)-propoxy]-pyridine-1-oxide-3-carboximidoyl chloride citrate (BRX-345), and N-[2-hydroxy-3-(1-piperidinyl)-propoxy]-pyridine-1-oxide-3-carboximidoyl chloride maleate (BRX-220).

In one embodiment the small molecule inducer of Hsp70 is selected from the group consisting of (+)-R—N-[2-hydroxy-3-(1-piperidinyl)-propoxy]-pyridine-1-oxide-3-carboximidoyl chloride citrate; (−)-S—N-[2-hydroxy-3-(1-piperidinyl)-propoxy]-pyridine-1-oxide-3-carboximidoyl chloride citrate; (+)-R—N-[2-hydroxy-3-(1-piperidinyl)-propoxy]-pyridine-1-oxide-3-carboximidoyl chloride maleate; and (−)-S—N-[2-hydroxy-3-(1-piperidinyl)-propoxy]-pyridine-1-oxide-3-carboximidoyl chloride maleate.

BGP-15

In one embodiment the small molecule inducer of Hsp70 is N-[2-hydroxy-3-(1-piperidinyl)propoxy]-3-pyridinecarboximidamide, dihydrochloride (BGP-15), its stereoisomers and the acid addition salts thereof.

In one embodiment the small molecule inducer of Hsp70 is selected from N-[2-hydroxy-3-(1-piperidinyl)propoxy]-3-pyridinecarboximidamide, dihydrochloride (BGP-15), its optically active (+) or (−) enantiomer, a mixture of the enantiomers of any ratio, and the racemic compound, furthermore, the acid addition salts formed from any of the above compounds with mineral or organic acids constitute objects of the present disclosure. All possible geometrical isomer forms of N-[2-hydroxy-3-(1-piperidinyl)propoxy]-3-pyridinecarboximidamide, dihydrochloride belong to the scope of the disclosure. The term “the stereoisomers of N-[2-hydroxy-3-(1-piperidinyl)propoxy]-3-pyridinecarboximidamide, dihydrochloride” refers to all possible optical and geometrical isomers of the compound.

Iroxanadine

In one embodiment the small molecule inducer of Hsp70 is selected from 5,6-dihydro-5-(1-piperidinyl)methyl-3-(3-pyridyl)-4H-1,2,4-oxadiazine (iroxanadine), its stereoisomers and the acid addition salts thereof. Iroxanadine is further described in e.g. WO 97/16439 and WO 00/35914.

In one embodiment the small molecule inducer of Hsp70 is selected from 5,6-dihydro-5-(1-piperidinyl)methyl-3-(3-pyridyl)-4H-1,2,4-oxadiazine (iroxanadine), its optically active (+) or (−) enantiomer, a mixture of the enantiomers of any ratio, and the racemic compound, furthermore, the acid addition salts formed from any of the above compounds with mineral or organic acids constitute objects of the present disclosure. All possible geometrical isomer forms of 5,6-dihydro-5-(1-piperidinyl)methyl-3-(3-pyridyl)-4H-1,2,4-oxadiazine belong to the scope of the disclosure. The term “the stereoisomers of 5,6-dihydro-5-(1-piperidinyl)methyl-3-(3-pyridyl)-4H-1,2,4-oxadiazine” refers to all possible optical and geometrical isomers of the compound.

If desired, the 5,6-dihydro-5-(1-piperidinyl)methyl-3-(3-pyridyl)-4H-1,2,4-oxadiazine or one of its optically active enantiomers can be transformed into an acid addition salt with a mineral or organic acid, by known methods.

In one embodiment the small molecule inducer of Hsp70 is the racemate of 5,6-dihydro-5-(1-piperidinyl)methyl-3-(3-pyridyl)-4H-1,2,4-oxadiazine.

In one embodiment the small molecule inducer of Hsp70 is an optically active stereoisomer of 5,6-dihydro-5-(1-piperidinyl)methyl-3-(3-pyridyl)-4H-1,2,4-oxadiazine.

In one embodiment the small molecule inducer of Hsp70 is an enantiomer of 5,6-dihydro-5-(1-piperidinyl)methyl-3-(3-pyridyl)-4H-1,2,4-oxadiazine.

In one embodiment the small molecule inducer of Hsp70 is selected from the group consisting of (+)-5,6-dihydro-5-(1-piperidinyl)methyl-3-(3-pyridyl)-4H-1,2,4-oxadiazine and (−)-5,6-dihydro-5-(1-piperidinyl)methyl-3-(3-pyridyl)-4H-1,2,4-oxadiazine.

In one embodiment the small molecule inducer of Hsp70 is an acid addition salt of 5,6-dihydro-5-(1-piperidinyl)methyl-3-(3-pyridyl)-4H-1,2,4-oxadiazine.

In one embodiment the small molecule inducer of Hsp70 is selected from the group consisting of 5,6-dihydro-5-(1-piperidinyl)methyl-3-(3-pyridyl)-4H-1,2,4-oxadiazine citrate, and 5,6-dihydro-5-(1-piperidinyl)methyl-3-(3-pyridyl)-4H-1,2,4-oxadiazine maleate.

In one embodiment the small molecule inducer of Hsp70 is selected from the group consisting of (+)-5,6-dihydro-5-(1-piperidinyl)methyl-3-(3-pyridyl)-4H-1,2,4-oxadiazine citrate; (−)-5,6-dihydro-5-(1-piperidinyl)methyl-3-(3-pyridyl)-4H-1,2,4-oxadiazine citrate; (+)-5,6-dihydro-5-(1-piperidinyl)methyl-3-(3-pyridyl)-4H-1,2,4-oxadiazine maleate; and (−)-5,6-dihydro-5-(1-piperidinyl)methyl-3-(3-pyridyl)-4H-1,2,4-oxadiazine maleate.

Bimoclomol

In one embodiment the small molecule inducer of Hsp70 is selected from N-[2-hydroxy-3-(1-piperidinyl)-propoxy]-3-pyridinecarboximidoyl chloride (bimoclomol) its stereoisomers and the acid addition salts thereof. Bimoclomol is further described in e.g. WO 1997/16439.

In one embodiment the small molecule inducer of Hsp70 is selected from N-[2-hydroxy-3-(1-piperidinyl)-propoxy]-3-pyridinecarboximidoyl chloride (bimoclomol), its optically active (+) or (−) enantiomer, a mixture of the enantiomers of any ratio, and the racemic compound, furthermore, the acid addition salts formed from any of the above compounds with mineral or organic acids constitute objects of the present disclosure. All possible geometrical isomer forms of N-[2-hydroxy-3-(1-piperidinyl)-propoxy]-3-pyridinecarboximidoyl chloride belong to the scope of the disclosure. The term “the stereoisomers of N-[2-hydroxy-3-(1-piperidinyl)-propoxy]-3-pyridinecarboximidoyl chloride” refers to all possible optical and geometrical isomers of the compound.

If desired, the N-[2-hydroxy-3-(1-piperidinyl)-propoxy]-3-pyridinecarboximidoyl chloride or one of its optically active enantiomers can be transformed into an acid addition salt with a mineral or organic acid, by known methods.

In one embodiment the small molecule inducer of Hsp70 is the racemate of N-[2-hydroxy-3-(1-piperidinyl)-propoxy]-3-pyridinecarboximidoyl chloride.

In one embodiment the small molecule inducer of Hsp70 is an optically active stereoisomer of N-[2-hydroxy-3-(1-piperidinyl)-propoxy]-3-pyridinecarboximidoyl chloride.

In one embodiment the small molecule inducer of Hsp70 is an enantiomer of N-[2-hydroxy-3-(1-piperidinyl)-propoxy]-3-pyridinecarboximidoyl chloride.

In one embodiment the small molecule inducer of Hsp70 is selected from the group consisting of (+)-R—N-[2-hydroxy-3-(1-piperidinyl)-propoxy]-3-pyridinecarboximidoyl chloride and (−)-(S)—N-[2-hydroxy-3-(1-piperidinyl)-propoxy]-3-pyridinecarboximidoyl chloride.

In one embodiment the small molecule inducer of Hsp70 is an acid addition salt of N-[2-hydroxy-3-(1-piperidinyl)-propoxy]-3-pyridinecarboximidoyl chloride.

In one embodiment the small molecule inducer of Hsp70 is selected from the group consisting of N-[2-hydroxy-3-(1-piperidinyl)-propoxy]-3-pyridinecarboximidoyl chloride citrate, and N-[2-hydroxy-3-(1-piperidinyl)-propoxy]-3-pyridinecarboximidoyl chloride maleate.

In one embodiment the small molecule inducer of Hsp70 is selected from the group consisting of (+)-R—N-[2-hydroxy-3-(1-piperidinyl)-propoxy]-3-pyridinecarboximidoyl chloride citrate; (−)-S—N-[2-hydroxy-3-(1-piperidinyl)-propoxy]-3-pyridinecarboximidoyl chloride citrate; (+)-R—N-[2-hydroxy-3-(1-piperidinyl)-propoxy]-3-pyridinecarboximidoyl chloride maleate; and (−)-S—N-[2-hydroxy-3-(1-piperidinyl)-propoxy]-3-pyridinecarboximidoyl chloride maleate.

Inducers for Treatment

In one embodiment there is provided a bioactive agent capable of increasing the intracellular concentration of Hsp70 by amplifying Hsp70 gene expression, wherein said bioactive agent is a hydroxylamine derivative,

wherein said bioactive agent is selected from the group consisting of:
N-[2-hydroxy-3-(1-piperidinyl)-propoxy]-pyridine-1-oxide-3-carboximidoyl chloride (arimoclomol), its stereoisomers and the acid addition salts thereof,
5,6-dihydro-5-(1-piperidinyl)methyl-3-(3-pyridyl)-4H-1,2,4-oxadiazine (iroxanadine), its stereoisomers and the acid addition salts thereof,
N-[2-hydroxy-3-(1-piperidinyl)-propoxy]-3-pyridinecarboximidoyl chloride (bimoclomol) its stereoisomers and the acid addition salts thereof, and
N-[2-hydroxy-3-(1-piperidinyl)propoxy]-3-pyridinecarboximidamide, dihydrochloride (BGP-15), its stereoisomers and the acid addition salts thereof,
   for use in the treatment of a frontotemporal disorder.

In one embodiment said frontotemporal disorder is associated with a mutation in the VCP gene, and/or is associated with one or more of TDP-43 mislocalisation, cytoplasmic ubiquitin aggregation, p-tau lesions, p62 and LC3 expression or aggregation, or stress granule formation.

In one embodiment there is provided a bioactive agent capable of increasing the intracellular concentration of Hsp70 by amplifying Hsp70 gene expression, wherein said bioactive agent is a hydroxylamine derivative,

wherein said bioactive agent is selected from the group consisting of:
N-[2-hydroxy-3-(1-piperidinyl)-propoxy]-pyridine-1-oxide-3-carboximidoyl chloride (arimoclomol), its stereoisomers and the acid addition salts thereof,
5,6-dihydro-5-(1-piperidinyl)methyl-3-(3-pyridyl)-4H-1,2,4-oxadiazine (iroxanadine), its stereoisomers and the acid addition salts thereof,
N-[2-hydroxy-3-(1-piperidinyl)-propoxy]-3-pyridinecarboximidoyl chloride (bimoclomol) its stereoisomers and the acid addition salts thereof, and
N-[2-hydroxy-3-(1-piperidinyl)propoxy]-3-pyridinecarboximidamide, dihydrochloride (BGP-15), its stereoisomers and the acid addition salts thereof,
   for use in the treatment of a frontotemporal disorder selected from the group consisting of frontotemporal lobar degeneration (FTLD), frontotemporal dementia (FTD), IBM with early-onset PDB and FTD (IBMPFD), inclusion body myopathy (IBM) with FTD, Paget's disease of bone (PDB) with FTD, IBMPFD with amyotrophic lateral sclerosis (ALS) (IBMPFD-ALS) and ALS-FTD.

In one embodiment there is provided a compound selected from the group consisting of (+)-R—N-[2-hydroxy-3-(1-piperidinyl)-propoxy]-pyridine-1-oxide-3-carboximidoyl chloride citrate; (−)-S—N-[2-hydroxy-3-(1-piperidinyl)-propoxy]-pyridine-1-oxide-3-carboximidoyl chloride citrate; (+)-R—N-[2-hydroxy-3-(1-piperidinyl)-propoxy]-pyridine-1-oxide-3-carboximidoyl chloride maleate; and (−)-S—N-[2-hydroxy-3-(1-piperidinyl)-propoxy]-pyridine-1-oxide-3-carboximidoyl chloride maleate, for use in the treatment of a frontotemporal disorder, such as a frontotemporal disorder selected from the group consisting of frontotemporal lobar degeneration (FTLD), frontotemporal dementia (FTD), IBM with early-onset PDB and FTD (IBMPFD), inclusion body myopathy (IBM) with FTD, Paget's disease of bone (PDB) with FTD, IBMPFD with amyotrophic lateral sclerosis (ALS) (IBMPFD-ALS) and ALS-FTD.

Other Inducers of Heat Shock Proteins

In one embodiment the bioactive agent is an inducer of Hsp70. Any means for inducing Hsp70 expression is envisioned to be encompassed herewith, some of which are outlined herein below.

In one embodiment the inducer of Hsp70 is sub-lethal heat therapy. Increasing the temperature of an individual is a potent inducer of HSPs including Hsp70, and as such sub-lethal heat therapy is a means for inducing Hsp70. In one embodiment, sub-lethal heat therapy comprises increasing the temperature of an individual to a core temperature of about 38° C., such as about 39° C., for example about 40° C., such as about 41° C., for example about 42° C., such as about 43° C.

Psychological stress such as predatory fear and electric shock can evoke a stress induced eHsp70 release, a process which is suggested to be dependent on cathecholamine signaling. Further, adrenaline and noradrenalin can evoke Hsp70 release.

A number of compounds have been shown to induce (or co-induce) HSPs, including Hsp70. In one embodiment the inducer of Hsp70 is selected from the group consisting of: membrane-interactive compounds such as alkyllysophospholipid edelfosine (ET-18-OCH3 or 1-octadecyl-2-methyl-rac-glycero-3-phosphocholine); anti-inflammatory drugs including cyclooxygenase 1/2 inhibitors such as celecoxib and rofecoxib, as well as NSAIDs such as acetyl-salicylic acid, sodium salicylate and indomethacin; dexamethasone; prostaglandins PGA1, PGj2 and 2-cyclopentene-1-one; peroxidase proliferator-activated receptor-gamma agonists; tubulin-interacting anticancer agents including vincristine and paclitaxel; the insulin sensitizer pioglitazone; anti-neoplastic agents such as carboplatin, doxorubicin, fludarabine, ifosfamide and cytarabine; Hsp90 inhibitors including geldanamycin, 17-AAG, 17-DMAG, radicicol, herbimycin-A and arachidonic acid; proteasome inhibitors such as MG132, lactacystin, Bortezomib, Carfilzomib and Oprozomib; serine protease inhibitors such as DCIC, TLCK and TPCK; Histone Deacetylase Inhibitors (HDACi) including SAHA/vorinostat, Belinostat/PXD101, LB-205, LBH589 (panobinostat), FK-228, CI-994, trichostatin A (TSA) and PCI-34051; anti-ulcer drugs including geranylgeranylacetone (GGA), rebamipide, carbenoxolone and polaprezinc (zinc L-carnosine); heavy metals (zinc and tin); cocaine; nicotine; alcohol; alpha-adrenergic agonists; cyclopentenone prostanoids; L-type Ca++ channel blockers, such as L-type Ca++ channel blockers that also inhibits ryanodine receptors, such as lacidipine; ryanodine receptor antagonists such as DHBP (1,1′-diheptyl-4,4′-bipyridium; as well as herbal medicines including paeoniflorin, glycyrrhizin, celastrol, dihydrocelastrol, dihydrocelastrol diacetate and curcumin.

In one embodiment the inducer of Hsp70 is a proteasome inhibitor. In one embodiment the proteasome inhibitor is selected from the group consisting of Bortezomib, Carfilzomib, Oprozomib, MG132 and lactacystin.

In one embodiment the inducer of Hsp70 is a HDAC inhibitor. In one embodiment the HDACi is selected form the group consisting of SAHA/vorinostat, Belinostat/PXD101, LB-205, LBH589 (panobinostat), FK-228, CI-994, trichostatin A (TSA) and PCI-34051.

Membrane Fluidizers

In one embodiment the inducer of Hsp70 is is a membrane fluidizer. Treatment with a membrane fluidizer may also be termed lipid therapy.

Besides the denaturation of a proportion of cellular proteins during heat (proteotoxicity), a change in the fluidity of membranes is also proposed as being a cellular thermo-sensor that initiates the heat shock response and induces HSPs. Indeed, chemically induced membrane perturbations—analogous with heat induced plasma membrane fluidization—are capable of activating HSP, without causing protein denaturation.

In one embodiment the inducer of Hsp70 is a membrane fluidizer selected from the group consisting of benzyl alcohol, heptanol, AL721, docosahexaenoic acid, aliphatic alcohols, oleyl alcohol, dimethylaminoethanol, A₂C, farnesol and anaesthetics such as lidocaine, ropivacaine, bupivacaine and mepivacaine, as well as others known to the skilled person.

Heat Shock Protein 70

It is also an aspect to provide Hsp70, or a functional fragment or variant thereof, for use in treating a frontotemporal disorder.

In one embodiment there is provided use of Hsp70, or a functional fragment or variant thereof, for the manufacture of a medicament for the treatment of frontotemporal disorder.

In one embodiment there is provided a method of treating a frontotemporal disorder, said method comprising one or more steps of administering Hsp70, or a functional fragment or variant thereof, to an individual in need thereof.

It is understood that Hsp70, or a functional fragment or variant thereof, as defined herein can be any natural or synthetic product, and may be produced by any conventional technique known to the person skilled in the art.

In one embodiment, Hsp70, or a functional fragment or variant thereof, is purified from a natural source. Said natural source may be any plant, animal or bacteria which expresses, or may be induced to express, Hsp70 in a form suitable for administering to an individual in need thereof.

In a particular embodiment, Hsp70, or a functional fragment or variant thereof, is made synthetically. It follows that Hsp70, or a functional fragment or variant thereof, in one embodiment is a recombinant protein made by conventional techniques and as such is denoted rHsp70.

The Hsp70 as defined herein, synthetic or natural, may have a sequence which is derived from any suitable species of plant, animal or bacteria. In one embodiment, said rHsp70 is derived from a mammal. Said mammal may be selected form the group consisting of human (Homo sapiens), mouse (Mus musculus), cow, dog, rat, ferret, pig, sheep, and monkey. In another embodiment, said rHsp70 is derived from bacteria.

Hsp70 is characterized in part by having a very high degree of interspecies sequence conservation, thus possibly allowing for Hsp70 derived from one species to be used in another species without eliciting a harmful immune response.

In one particular embodiment, said rHsp70 has a sequence derived from human Hsp70.

In one particular embodiment, said rHsp70 has a sequence derived from more than one species. Said Hsp70, or a functional fragment or variant thereof, may thus in one embodiment be a chimera.

In one embodiment Hsp70 is meant to denote any of the two inducible Hsp70 family members with loci names HSPA1A and HSPA1B.

In one embodiment said Hsp70 is selected from HSPA1A (SEQ ID NOs:1 and 2) and HSPA1B (SEQ ID NOs:4 and 5), or a functional fragment or variant thereof. In SEQ ID NO:2 the initiator methionine (M at position 1) of SEQ ID NO:1 is removed. In SEQ ID NO:5 the initiator methionine (M at position 1) of SEQ ID NO:4 is removed. In vivo this occurs by post-translational processing.

In one embodiment, the Hsp70 is selected from any one of SEQ ID NO:s 1, 2, 4 and 5, or functional fragments or variants thereof, including any naturally occurring variants thereof, such as variants derived from molecule processing and/or amino acid modifications (including any acetylation, phosphorylation and methylation).

In one embodiment, the Hsp70 protein has 100% identity to wild-type Hsp70 protein. In another embodiment, the Hsp70 protein has less than 100% identity to the wild-type Hsp70 protein, such as 99.9 to 95% identity, for example 95 to 90% identity, such as 90 to 85% identity, for example 85 to 80% identity, such as 80 to 75% identity, for example 75 to 60% identity to the wild-type protein. Regardless of the degree of identity, any fragment or variant of Hsp70 that retains its relevant biological effects is encompassed herewith.

In one embodiment said variant of Hsp70 has 99.9 to 99% identity, for example 99 to 98% identity, such as 98 to 97% identity, for example 97 to 96% identity, such as 96 to 95% identity, for example 95 to 94% identity, such as 94 to 93% identity, for example 93 to 92% identity, such as 92 to 91% identity, for example 91 to 90% identity, such as 90 to 85% identity, for example 85 to 80% identity, such as 80 to 75% identity, for example 75 to 70% identity, such as 70 to 65% identity, for example 65 to 60% identity to Hsp70 selected from HSPA1A (SEQ ID NOs:1 and 2) and HSPA1B (SEQ ID NOs: 4 and 5), or a fragment thereof.

In one embodiment, the bioactive agent is Hsp70. In one embodiment, said Hsp70 is full length Hsp70. In one embodiment said Hsp70 is HSPA1A, or a functional fragment or variant thereof. In one embodiment said Hsp70 is SEQ ID NO:1 or 2, or a functional fragment or variant thereof.

It is also an embodiment to provide a functional fragment or variant of Hsp70. As defined herein, a functional fragment or variant is any fragment or variant of Hsp70 which retains the capability of one or more of:
i) reducing cytoplasmic ubiquitin aggregation,
ii) reducing Transactive response DNA binding protein 43 kDa (TDP-43) cellular mislocalisation,
iii) reducing motor unit loss,
iv) reducing stress granule formation, such as reducing stress granule markers including Tia1, FMRP and G3BP,
v) reducing p-tau positive lesions, and
vi) reducing P62 and/or LC3 expression or cytoplasmic aggregation.

In one embodiment, the bioactive agent is a functional fragment or variant of Hsp70.

In one embodiment, the bioactive agent is a functional fragment or variant of Hsp70, in which Hsp70 is modified by one or more deletion(s), addition(s) or substitution(s) of the wild type Hsp70.

In one embodiment, the bioactive agent is a naturally occurring variant of Hsp70, or a fragment of a naturally occurring variant of Hsp70.

In one embodiment a variant of Hsp70 comprises one or more of D-A at position 10, E-D at position 110, D-A at position 199, K-R at position 561, N-acetylalanine at position 2, N6-acetyllysine at position 108, N6-acetyllysine at position 246, N6-acetyllysine at position 348, N6,N6,N6-trimethyllysine at position 561, phosphoserine at position 631, phosphoserine at position 633 and phosphothreonine at position 636. In one embodiment a naturally occurring variant of Hsp70 is Isoform 1 wherein amino acids of position 96-150 are missing (PODMV8-2).

In one embodiment, a functional fragment or variant of Hsp70 is a variant of Hsp70 in which one or more amino acids has been substituted (or mutated). Said substitution(s) comprises equivalent or conservative substitution(s), or a non-equivalent or non-conservative substitution(s). The term Hsp70 and variants thereof also embraces post-translational modifications introduced by chemical or enzyme-catalyzed reactions, as are known in the art, and chemical modifications such as ubiquitination, labeling, pegylation, glycosylation, amidation, alkylation and esterification. In one embodiment said Hsp70 has been post-translationally modified, including including acetylation, phosphorylation and methylation at any position.

In one embodiment 0.1 to 1% of the amino acid residues of wild type Hsp70 has been substituted, such as 1 to 2%, for example 2 to 3%, such as 3 to 4%, for example 4 to 5%, such as 5 to 10%, for example 10 to 15%, such as 15 to 20%, for example 20 to 30%, such as 30 to 40%, for example 40 to 50%, such as 50 to 60%, for example 60 to 70%, such as 70 to 80%, for example 80 to 90%, such as 90 to 100% amino acid residues.

In one embodiment 1-2, 2-3, 3-4, 4-5 of the amino acid residues of wild type Hsp70 has been substituted, such as 5 to 10, for example 10 to 15, such as 15 to 20, for example 20 to 30, such as 30 to 40, for example 40 to 50, such as 50 to 75, for example 75 to 100, such as 100 to 150, for example 150 to 200, such as 200 to 300, for example 300 to 400, such as 400 to 500 amino acid residues.

In one embodiment, the Hsp70 or functional fragment or variant of Hsp70 is a fusion protein. In one embodiment, said Hsp70 or functional fragment or variant of Hsp70 is fused to a tag.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

The classification of equivalent amino acids refers in one embodiment to the following classes: 1) HRK, 2) DENQ, 3) C, 4) STPAG, 5) MILV and 6) FYW Within the meaning of the term “equivalent amino acid substitution” as applied herein, one amino acid may be substituted for another, in one embodiment, within the groups of amino acids indicated herein below:
i) Amino acids having polar side chains (Asp, Glu, Lys, Arg, His, Asn, Gin, Ser, Thr, Tyr, and Cys,)
ii) Amino acids having non-polar side chains (Gly, Ala, Val, Leu, lie, Phe, Trp, Pro, and Met)
iii) Amino acids having aliphatic side chains (Gly, Ala Val, Leu, lie)
iv) Amino acids having cyclic side chains (Phe, Tyr, Trp, His, Pro)
v) Amino acids having aromatic side chains (Phe, Tyr, Trp)
vi) Amino acids having acidic side chains (Asp, Glu)
vii) Amino acids having basic side chains (Lys, Arg, His)
viii) Amino acids having amide side chains (Asn, Gin)
ix) Amino acids having hydroxy side chains (Ser, Thr)
x) Amino acids having sulphor-containing side chains (Cys, Met),
xi) Neutral, weakly hydrophobic amino acids (Pro, Ala, Gly, Ser, Thr)
xii) Hydrophilic, acidic amino acids (Gln, Asn, Glu, Asp), and
xiii) Hydrophobic amino acids (Leu, lie, Val)

The wild type Hsp70 protein has a total length of 641 amino acids (640 amino acids after removal of initiator methionine at position 1). A fragment of Hsp70 is in one embodiment meant to comprise any fragment with a total length of less than the wild type protein, such as having a total length of is 5 to 25 amino acids, such as 25 to 50 amino acids, for example 50 to 75 amino acids, such as 75 to 100 amino acids, for example 100 to 125 amino acids, such as 125 to 150 amino acids, for example 150 to 175 amino acids, such as 175 to 200 amino acids, for example 200 to 225 amino acids, such as 225 to 250 amino acids, for example 250 to 275 amino acids, such as 275 to 300 amino acids, for example 300 to 325 amino acids, such as 325 to 350 amino acids, for example 350 to 375 amino acids, such as 375 to 400 amino acids, for example 400 to 425 amino acids, such as 425 to 450 amino acids, for example 450 to 475 amino acids, such as 475 to 500 amino acids, for example 500 to 525 amino acids, such as 525 to 550 amino acids, for example 550 to 575 amino acids, such as 575 to 600 amino acids, for example 600 to 625 amino acids, such as 625 to 640 amino acids derived from Hsp70.

A fragment of Hsp70 is in one embodiment a truncated version of the wild type protein. A fragment may be truncated by shortening of the protein from either the amino-terminal or the carboxy-terminal ends of the protein, or it may be truncated by deletion of one or more internal regions of any size of the protein.

In one embodiment the Hsp70 is a variant of a fragment, i.e. a fragment of Hsp70 as defined herein wherein one or more amino acids are substituted as defined herein.

It is appreciated that the exact quantitative effect of the functional fragment or variant may be different from the effect of the full-length molecule. In some instances, the functional fragment or variant may indeed be more effective than the full-length molecule.

The present disclosure also relates to variants of Hsp70, or fragments thereof, wherein the substitutions have been designed by computational analysis that uses sequence homology to predict whether a substitution affects protein function (e.g. Pauline C. Ng and Steven Henikoff, Genome Research, Vol. 11, Issue 5, 863-874, May 2001).

Ectopic Expression of Hsp70

In one embodiment, Hsp70, or a functional fragment or variant thereof, is expressed from a vector. In one embodiment Hsp70, or a functional fragment or variant thereof, is administered to an individual in need thereof in the form of a vector.

The vector used for expressing Hsp70, or a functional fragment or variant thereof, is in one embodiment selected from the group consisting of: viral vectors (retroviral and adenoviral) or non-viral vectors (e.g. plasmid, cosmid, bacteriophage).

In one embodiment, said vector comprises one or more of an origin of replication, a marker for selection and one or more recognition sites for a restriction endonuclease. In another embodiment, said vector is operably linked to regulatory sequences controlling the transcription of said Hsp70, or a functional fragment or variant thereof, in a suitable host cell.

In one embodiment there is provided a method for producing Hsp70, or a functional fragment or variant thereof, as described herein; said method comprising the steps of providing a vector encoding said Hsp70, or a functional fragment or variant thereof, and expressing said vector either in vitro, or in vivo in a suitable host organism, thereby producing said Hsp70, or a functional fragment or variant thereof.

In one embodiment there is provided an isolated recombinant or transgenic host cell comprising a vector encoding Hsp70, or a functional fragment or variant thereof, as defined herein.

In one embodiment there is provided a method for generating a recombinant or transgenic host cell, said method comprising the steps of providing a vector encoding Hsp70, or a functional fragment or variant thereof, introducing said vector into said recombinant or transgenic host cell and optionally also expressing said vector in said recombinant or transgenic host cell, thereby generating a recombinant or transgenic host cell producing said Hsp70, or a functional fragment or variant thereof.

In another embodiment there is provided a transgenic, mammalian organism comprising the host cell producing said Hsp70, or a functional fragment or variant thereof. In a further embodiment, the transgenic, mammalian organism comprising the recombinant or transgenic host cell according to the present disclosure is non-human. The transgenic host cell can be selected from the group consisting of a mammalian, plant, bacterial, yeast or fungal host cell.

To improve the delivery of the DNA into the cell, the DNA must be protected from damage and its entry into the cell must be facilitated. Lipoplexes and polyplexes, have been created that have the ability to protect the DNA from undesirable degradation during the transfection process. Plasmid DNA can be covered with lipids in an organized structure like a micelle or a liposome. When the organized structure is complexed with DNA it is called a lipoplex. There are three types of lipids that may be employed for forming liposomes; anionic (negatively charged), neutral, or cationic (positively charged). Complexes of polymers with DNA are called polyplexes. Most polyplexes consist of cationic polymers and their production is regulated by ionic interactions.

In one embodiment, the vector comprising Hsp70, or a functional fragment or variant thereof, may be used for gene therapy. Gene therapy is the insertion of genes into an individual's cells and tissues to treat a disease, such as a hereditary disease in which a deleterious mutant allele is replaced with a functional one.

In another embodiment, Hsp70, or a functional fragment or variant thereof, may be administered as naked DNA. This is the simplest form of non-viral transfection. Delivery of naked DNA may be performed by use of electroporation, sonoporation, or the use of a “gene gun”, which shoots DNA coated gold particles into a cell using high pressure gas.

Composition

Whilst it is possible for the bioactive agents to be administered as the raw chemical, it is in some embodiments preferred to present them in the form of a pharmaceutical formulation. Accordingly, also provided herewith is a composition, such as a pharmaceutical composition, i.e. a pharmaceutically safe composition, comprising a bioactive agent as defined herein. The composition in one embodiment comprises a pharmaceutically and/or physiologically acceptable carriers or excipients.

Pharmaceutical compositions containing a bioactive agent of the present disclosure may be prepared by conventional techniques, e.g. as described in Remington: The Science and Practice of Pharmacy, 20^{th} Edition, Gennaro, Ed., Mack Publishing Co., Easton, Pa., 2000.

It is thus an aspect to provide a composition, such as a pharmaceutical composition, comprising a bioactive agent that increases the intracellular concentration and/or activity of one or more heat shock proteins, including Hsp70, for use in the treatment of a frontotemporal disorder.

Administration and Dosage

A bioactive agent or composition comprising the same as defined herein is in one embodiment administered to individuals in need thereof in pharmaceutically effective doses or a therapeutically effective amount.

A therapeutically effective amount of a bioactive agent is in one embodiment an amount sufficient to cure, prevent, reduce the risk of, alleviate or partially arrest the clinical manifestations of a given disease or disorder and its complications. The amount that is effective for a particular therapeutic purpose will depend on the severity and the sort of the disorder as well as on the weight and general state of the subject. An amount adequate to accomplish this is defined as a “therapeutically effective amount”.

In one embodiment, the composition is administered in doses of 1 μg/day to 100 mg/day; such as 1 μg/day to 10 μg/day, such as 10 μg/day to 100 μg/day, such as 100 μg/day to 250 μg/day, such as 250 μg/day to 500 μg/day, such as 500 μg/day to 750 μg/day, such as 750 μg/day to 1 mg/day, such as 1 mg/day to 2 mg/day, such as 2 mg/day to 5 mg/day, or such as 5 mg/day to 10 mg/day, such as 10 mg/day to 20 mg/day, such as 20 mg/day to 30 mg/day, such as 30 mg/day to 40 mg/day, such as 40 mg/day to 50 mg/day, such as 50 mg/day to 75 mg/day, or such as 75 mg/day to 100 mg/day, such as 100 mg/day to 150 mg/day, such as 150 mg/day to 200 mg/day, or such as 200 mg/day to 250 mg/day, such as 250 mg/day to 300 mg/day, such as 300 mg/day to 400 mg/day, such as 400 mg/day to 500 mg/day, such as 500 mg/day to 600 mg/day, such as 600 mg/day to 700 mg/day, such as 700 mg/day to 800 mg/day, such as 800 mg/day to 900 mg/day, such as 900 mg/day to 1000 mg/day.

In one embodiment, the bioactive agent or composition is administered at a dose of 1 μg/kg body weight to 100 mg/kg body weight; such as 1 to 10 μg/kg body weight, such as 10 to 100 μg/day, such as 100 to 250 μg/kg body weight, such as 250 to 500 μg/kg body weight, such as 500 to 750 μg/kg body weight, such as 750 μg/kg body weight to 1 mg/kg body weight, such as 1 mg/kg body weight to 2 mg/kg body weight, such as 2 to 5 mg/kg body weight, such as 5 to 10 mg/kg body weight, such as 10 to 20 mg/kg body weight, such as 20 to 30 mg/kg body weight, such as 30 to 40 mg/kg body weight, such as 40 to 50 mg/kg body weight, such as 50 to 75 mg/kg body weight, or such as 75 to 100 mg/kg body weight.

In one embodiment, a dose is administered one or several times per day, such as from 1 to 6 times per day, such as from 1 to 5 times per day, such as from 1 to 4 times per day, such as from 1 to 3 times per day, such as from 1 to 2 times per day, such as from 2 to 4 times per day, such as from 2 to 3 times per day. In one embodiment, a dose is administered less than once a day, such as once every second day or once a week.

Routes of Administration

It will be appreciated that the preferred route of administration will depend on the general condition and age of the subject to be treated, the nature of the condition to be treated, the location of the tissue to be treated in the body and the active ingredient chosen.

Systemic Treatment

In one embodiment, the route of administration allows for introducing the bioactive agent into the blood stream to ultimately target the sites of desired action.

In one embodiment the routes of administration is any suitable route, such as an enteral route (including the oral, rectal, nasal, pulmonary, buccal, sublingual, transdermal, intracisternal and intraperitoneal administration), and/or a parenteral route (including subcutaneous, intramuscular, intrathecal, intravenous and intradermal administration).

Appropriate dosage forms for such administration may be prepared by conventional techniques.

Parenteral Administration

Parenteral administration is any administration route not being the oral/enteral route whereby the bioactive agent avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

Accordingly, the bioactive agent or composition is in one embodiment administered topically to cross any mucosal membrane of an animal, e.g. in the nose, vagina, eye, mouth, genital tract, lungs, gastrointestinal tract, or rectum, for example the mucosa of the nose, or mouth, and accordingly, parenteral administration may also include buccal, sublingual, nasal, rectal, vaginal and intraperitoneal administration as well as pulmonal and bronchial administration by inhalation or installation. In some embodiments, the bioactive agent is administered topically to cross the skin.

In one embodiment, the intravenous, subcutaneous and intramuscular forms of parenteral administration are employed.

Local Treatment

In one embodiment, the bioactive agent or composition is used as a local treatment, i.e. is introduced directly to the site(s) of action. Accordingly, the bioactive agent may be applied to the skin or mucosa directly, or the bioactive agent may be injected into the site of action, for example into the diseased tissue or to an end artery leading directly to the diseased tissue.

Combination Treatment

It is also an aspect to provide a bioactive agent that increases the intracellular concentration and/or activity of one or more heat shock proteins, including Hsp70, for use in the treatment of a frontotemporal disorder, in combination with other treatment modalities.

Thus, in one embodiment, the bioactive agent is administered to an individual in need thereof in combination with at least one other treatment modality, such as conventional or known treatment modalities for frontotemporal disorders

Administering more than one treatment modality in combination may occur either simultaneously, or sequentially. Simultaneous administration may be two compounds comprised in the same composition or comprised in separate compositions, or may be one composition and one other treatment modality performed essentially at the same time. Sequential administration means that the more than one treatment modalities are administered at different time points, such as administering one treatment modality first, and administering the second treatment modality subsequently. The time frame for administering more than one treatment modality sequentially may be determined by a skilled person in the art for achieving the optimal effect, and may in one embodiment be between 30 minutes to 72 hours.

The treatment modalities in the form of chemical compounds may be administered together or separately, each at its most effective dosage. Administering more than one compound may have a synergistic effect, thus effectively reducing the required dosage of each drug.

It is also an aspect to provide a composition comprising, separately or together, i) a bioactive agent that increases the intracellular concentration and/or activity of one or more heat shock proteins, including Hsp70, and ii) other treatment modalities, for use in the treatment of a frontotemporal disorder.

In one embodiment other treatment modalities, or conventional or known treatment modalities for frontotemporal disorders.

In one embodiment the bioactive agent that increases the intracellular concentration and/or activity of one or more heat shock proteins, including Hsp70, is administered in combination with, and/or formulated as a combination product, with one or more further active ingredients.

Sequences

SEQ ID NO: 1: The protein sequence for Homo sapiens heat shock 70 kDa
protein 1A (HSPA1A_HUMAN) (NM_005345.5/UniProtKB-P0DMV8):
MAKAAAIGIDLGTTYSCVGVFQHGKVEIIANDQGNRTTPSYVAFTDTERLIGDAAKNQVALNPQNTVFDA
KRLIGRKFGDPVVQSDMKHWPFQVINDGDKPKVQVSYKGETKAFYPEEISSMVLTKMKEIAEAYLGYPVT
NAVITVPAYFNDSQRQATKDAGVIAGLNVLRIINEPTAAAIAYGLDRTGKGERNVLIFDLGGGTFDVSIL
TIDDGIFEVKATAGDTHLGGEDFDNRLVNHFVEEFKRKHKKDISQNKRAVRRLRTACERAKRTLSSSTQA
SLEIDSLFEGIDFYTSITRARFEELCSDLFRSTLEPVEKALRDAKLDKAQIHDLVLVGGSTRIPKVQKLL
QDFFNGRDLNKSINPDEAVAYGAAVQAAILMGDKSENVQDLLLLDVAPLSLGLETAGGVMTALIKRNSTI
PTKQTQIFTTYSDNQPGVLIQVYEGERAMTKDNNLLGRFELSGIPPAPRGVPQIEVTFDIDANGILNVTA
TDKSTGKANKITITNDKGRLSKEEIERMVQEAEKYKAEDEVQRERVSAKNALESYAFNMKSAVEDEGLKG
KISEADKKKVLDKCQEVISWLDANTLAEKDEFEHKRKELEQVCNPIISGLYQGAGGPGPGGFGAQGPKGG
SGSGPTIEEVD
SEQ ID NO: 2: The initiator methionine (M at position 1) of SEQ ID NO:
1 is removed to yield a 640-amino acid long sequence (position 2-641):
AKAAAIGIDLGTTYSCVGVFQHGKVEIIANDQGNRTTPSYVAFTDTERLIGDAAKNQVALNPQNTVFDAK
RLIGRKFGDPVVQSDMKHWPFQVINDGDKPKVQVSYKGETKAFYPEEISSMVLTKMKEIAEAYLGYPVTN
AVITVPAYFNDSQRQATKDAGVIAGLNVLRIINEPTAAAIAYGLDRTGKGERNVLIFDLGGGTFDVSILT
IDDGIFEVKATAGDTHLGGEDFDNRLVNHFVEEFKRKHKKDISQNKRAVRRLRTACERAKRTLSSSTQAS
LEIDSLFEGIDFYTSITRARFEELCSDLFRSTLEPVEKALRDAKLDKAQIHDLVLVGGSTRIPKVQKLLQ
DFFNGRDLNKSINPDEAVAYGAAVQAAILMGDKSENVQDLLLLDVAPLSLGLETAGGVMTALIKRNSTIP
TKQTQIFTTYSDNQPGVLIQVYEGERAMTKDNNLLGRFELSGIPPAPRGVPQIEVTFDIDANGILNVTAT
DKSTGKANKITITNDKGRLSKEEIERMVQEAEKYKAEDEVQRERVSAKNALESYAFNMKSAVEDEGLKGK
ISEADKKKVLDKCQEVISWLDANTLAEKDEFEHKRKELEQVCNPIISGLYQGAGGPGPGGFGAQGPKGGS
GSGPTIEEVD
SEQ ID NO: 3: The nucleic acid (DNA) sequence for Homo sapiens heat
shock 70 kDa protein 1A (HSPA1A) (NM_005345.5):

| | |
|---|---|
| 1 | ataaaagccc aggggcaagc ggtccggata acggctagcc tgaggagctg ctgcgacagt |
| 61 | ccactacctt tttcgagagt gactcccgtt gtcccaaggc ttcccagagc gaacctgtgc |
| 121 | ggctgcaggc accggcgcgt cgagtttccg gcgtccggaa ggaccgagct cttctcgcgg |
| 181 | atccagtgtt ccgtttccag cccccaatct cagagcggag ccgacagaga gcagggaacc |
| 241 | ggcatggcca aagccgcggc gatcggcatc gacctgggca ccacctactc ctgcgtgggg |
| 301 | gtgttccaac acggcaaggt ggagatcatc gccaacgacc agggcaaccg caccaccccc |
| 361 | agctacgtgg ccttcacgga caccgagcgg ctcatcgggg atgcggccaa gaaccaggtg |
| 421 | gcgctgaacc cgcagaacac cgtgtttgac gcgaagcggc tgattggccg caagttcggc |
| 481 | gacccggtgg tgcagtcgga catgaagcac tggcctttcc aggtgatcaa cgacggagac |
| 541 | aagcccaagg tgcaggtgag ctacaagggg gagaccaagg cattctaccc cgaggagatc |
| 601 | tcgtccatgg tgctgaccaa gatgaaggag atcgccgagg cgtacctggg ctacccggtg |
| 661 | accaacgcgg tgatcaccgt gccggcctac ttcaacgact cgcagcgcca ggccaccaag |
| 721 | gatgcgggtg tgatcgcggg gctcaacgtg ctgcggatca tcaacgagcc cacggccgcc |
| 781 | gccatcgcct acggcctgga cagaacgggc aagggggagc gcaacgtgct catctttgac |
| 841 | ctgggcgggg gcaccttcga cgtgtccatc ctgacgatcg acgacggcat cttcgaggtg |
| 901 | aaggccacgg ccggggacac ccacctgggt ggggaggact ttgacaacag gctggtgaac |
| 961 | cacttcgtgg aggagttcaa gagaaaacac aagaaggaca tcagccagaa caagcgagcc |
| 1021 | gtgaggcggc tgcgcaccgc ctgcgagagg gccaagagga ccctgtcgtc cagcacccag |
| 1081 | gccagcctgg agatcgactc cctgtttgag ggcatcgact tctacacgtc catcaccagg |
| 1141 | gcgaggttcg aggagctgtg ctccgacctg ttccgaagca ccctggagcc cgtggagaag |
| 1201 | gctctgcgcg acgccaagct ggacaaggcc cagattcacg acctggtcct ggtcgggggc |
| 1261 | tccacccgca tccccaaggt gcagaagctg ctgcaggact tcttcaacgg gcgcgacctg |
| 1321 | aacaagagca tcaaccccga cgaggctgtg gcctacgggg cggcggtgca ggcggccatc |
| 1381 | ctgatggggg acaagtccga gaacgtgcag gacctgctgc tgctggacgt ggctcccctg |
| 1441 | tcgctggggc tggagacggc cggaggcgtg atgactgccc tgatcaagcg caactccacc |
| 1501 | atccccacca agcagacgca gatcttcacc acctactccg acaaccaacc cggggtgctg |
| 1561 | atccaggtgt acgagggcga gagggccatg acgaaagaca acaatctgtt ggggcgcttc |
| 1621 | gagctgagcg gcatccctcc ggcccccagg ggcgtgcccc agatcgaggt gaccttcgac |
| 1681 | atcgatgcca acggcatcct gaacgtcacg gccacggaca agagcaccgg caaggccaac |
| 1741 | aagatcacca tcaccaacga caagggccgc ctgagcaagg aggagatcga gcgcatggtg |
| 1801 | caggaggcgg agaagtacaa agcggaggac gaggtgcagc gcgagagggt gtcagccaag |
| 1861 | aacgccctgg agtcctacgc cttcaacatg aagagcgccg tggaggatga ggggctcaag |
| 1921 | ggcaagatca gcgaggcgga caagaagaag gtgctggaca agtgtcaaga ggtcatctcg |
| 1981 | tggctggacg ccaacacctt ggccgagaag gacgagtttg agcacaagag gaaggagctg |
| 2041 | gagcaggtgt gtaaccccat catcagcgga ctgtaccagg gtgccggtgg tcccgggcct |
| 2101 | gggggcttcg gggctcaggg tcccaaggga gggtctgggt caggccccac cattgaggag |
| 2161 | gtagattagg ggcctttcca agattgctgt ttttgttttg gagcttcaag actttgcatt |
| 2221 | tcctagtatt tctgtttgtc agttctcaat ttcctgtgtt tgcaatgttg aaattttttg |
| 2281 | gtgaagtact gaacttgctt tttttccggt ttctacatgc agagatgaat ttatactgcc |
| 2341 | atcttacgac tatttcttct ttttaataca cttaactcag gccatttttt aagttggtta |
| 2401 | cttcaaagta aataaacttt aaaattcaaa aaaaaaaaaa aaaaa |

SEQ ID NO: 4: The protein sequence for Homo sapiens heat shock 70 kDa
protein 1B (HSPA1B_HUMAN) (NM_005346.4/UniProtKB-P0DMV9):
MAKAAAIGIDLGTTYSCVGVFQHGKVEIIANDQGNRTTPSYVAFTDTERLIGDAAKNQVALNPQNTVFDA
KRLIGRKFGDPVVQSDMKHWPFQVINDGDKPKVQVSYKGETKAFYPEEISSMVLTKMKEIAEAYLGYPVT
NAVITVPAYFNDSQRQATKDAGVIAGLNVLRIINEPTAAAIAYGLDRTGKGERNVLIFDLGGGTFDVSIL
TIDDGIFEVKATAGDTHLGGEDFDNRLVNHFVEEFKRKHKKDISQNKRAVRRLRTACERAKRTLSSSTQA
SLEIDSLFEGIDFYTSITRARFEELCSDLFRSTLEPVEKALRDAKLDKAQIHDLVLVGGSTRIPKVQKLL
QDFFNGRDLNKSINPDEAVAYGAAVQAAILMGDKSENVQDLLLLDVAPLSLGLETAGGVMTALIKRNSTI
PTKQTQIFTTYSDNQPGVLIQVYEGERAMTKDNNLLGRFELSGIPPAPRGVPQIEVTFDIDANGILNVTA
TDKSTGKANKITITNDKGRLSKEEIERMVQEAEKYKAEDEVQRERVSAKNALESYAFNMKSAVEDEGLKG
KISEADKKKVLDKCQEVISWLDANTLAEKDEFEHKRKELEQVCNPIISGLYQGAGGPGPGGFGAQGPKGG
SGSGPTIEEVD
SEQ ID NO: 5: The initiator methionine (M at position 1) of SEQ ID NO:
4 is removed to yield a 640-amino acid long sequence (position 2-641):
AKAAAIGIDLGTTYSCVGVFQHGKVEIIANDQGNRTTPSYVAFTDTERLIGDAAKNQVALNPQNTVFDAK
RLIGRKFGDPVVQSDMKHWPFQVINDGDKPKVQVSYKGETKAFYPEEISSMVLTKMKEIAEAYLGYPVTN
AVITVPAYFNDSQRQATKDAGVIAGLNVLRIINEPTAAAIAYGLDRTGKGERNVLIFDLGGGTFDVSILT
IDDGIFEVKATAGDTHLGGEDFDNRLVNHFVEEFKRKHKKDISQNKRAVRRLRTACERAKRTLSSSTQAS
LEIDSLFEGIDFYTSITRARFEELCSDLFRSTLEPVEKALRDAKLDKAQIHDLVLVGGSTRIPKVQKLLQ
DFFNGRDLNKSINPDEAVAYGAAVQAAILMGDKSENVQDLLLLDVAPLSLGLETAGGVMTALIKRNSTIP
TKQTQIFTTYSDNQPGVLIQVYEGERAMTKDNNLLGRFELSGIPPAPRGVPQIEVTFDIDANGILNVTAT
DKSTGKANKITITNDKGRLSKEEIERMVQEAEKYKAEDEVQRERVSAKNALESYAFNMKSAVEDEGLKGK
ISEADKKKVLDKCQEVISWLDANTLAEKDEFEHKRKELEQVCNPIISGLYQGAGGPGPGGFGAQGPKGGS
GSGPTIEEVD
SEQ ID NO: 6 The nucleic acid (DNA) sequence for Homo sapiens heat
shock 70 kDa protein 1B (HSPA1B) (NM_005346.4):

| | |
|---|---|
| 1 | ggaaaacggc cagcctgagg agctgctgcg agggtccgct tcgtctttcg agagtgactc |
| 61 | ccgcggtccc aaggctttcc agagcgaacc tgtgcggctg caggcaccgg cgtgttgagt |
| 121 | ttccggcgtt ccgaaggact gagctcttgt cgcggatccc gtccgccgtt tccagccccc |
| 181 | agtctcagag cggagcccac agagcagggc accggcatgg ccaaagccgc ggcgatcggc |
| 241 | atcgacctgg gcaccaccta ctcctgcgtg ggggtgttcc aacacggcaa ggtggagatc |
| 301 | atcgccaacg accagggcaa ccgcaccacc cccagctacg tggccttcac ggacaccgag |
| 361 | cggctcatcg gggatgcggc caagaaccag gtggcgctga acccgcagaa caccgtgttt |
| 421 | gacgcgaagc ggctgatcgg ccgcaagttc ggcgacccgg tggtgcagtc ggacatgaag |
| 481 | cactggcctt tccaggtgat caacgacgga gacaagccca aggtgcaggt gagctacaag |
| 541 | ggggagacca aggcattcta ccccgaggag atctcgtcca tggtgctgac caagatgaag |
| 601 | gagatcgccg aggcgtacct gggctacccg gtgaccaacg cggtgatcac cgtgccggcc |
| 661 | tacttcaacg actcgcagcg ccaggccacc aaggatgcgg gtgtgatcgc ggggctcaac |
| 721 | gtgctgcgga tcatcaacga gcccacggcc gccgccatcg cctacggcct ggacagaacg |
| 781 | ggcaaggggg agcgcaacgt gctcatcttt gacctgggcg ggggcacctt cgacgtgtcc |
| 841 | atcctgacga tcgacgacgg catcttcgag gtgaaggcca cggccgggga cacccacctg |
| 901 | ggtggggagg actttgacaa caggctggtg aaccacttcg tggaggagtt caagagaaaa |
| 961 | cacaagaagg acatcagcca gaacaagcga gccgtgaggc ggctgcgcac cgcctgcgag |
| 1021 | agggccaaga ggaccctgtc gtccagcacc caggccagcc tggagatcga ctccctgttt |
| 1081 | gagggcatcg acttctacac gtccatcacc agggcgaggt tcgaggagct gtgctccgac |
| 1141 | ctgttccgaa gcaccctgga gcccgtggag aaggctctgc gcgacgccaa gctggacaag |
| 1201 | gcccagattc acgacctggt cctggtcggg ggctccaccc gcatccccaa ggtgcagaag |
| 1261 | ctgctgcagg acttcttcaa cgggcgcgac ctgaacaaga gcatcaaccc cgacgaggct |
| 1321 | gtggcctacg gggcggcggt gcaggcggcc atcctgatgg gggacaagtc cgagaacgtg |
| 1381 | caggacctgc tgctgctgga cgtggctccc ctgtcgctgg ggctggagac ggccggaggc |
| 1441 | gtgatgactg ccctgatcaa gcgcaactcc accatcccca ccaagcagac gcagatcttc |
| 1501 | accacctact ccgacaacca acccggggtg ctgatccagg tgtacgaggg cgagagggcc |
| 1561 | atgacgaaag acaacaatct gttggggcgc ttcgagctga gcggcatccc tccggccccc |
| 1621 | aggggcgtgc cccagatcga ggtgaccttc gacatcgatg ccaacggcat cctgaacgtc |
| 1681 | acggccacgg acaagagcac cggcaaggcc aacaagatca ccatcaccaa cgacaagggc |
| 1741 | cgcctgagca aggaggagat cgagcgcatg gtgcaggagg cggagaagta caaagcggag |
| 1801 | gacgaggtgc agcgcgagag ggtgtcagcc aagaacgccc tggagtccta cgccttcaac |
| 1861 | atgaagagcg ccgtggagga tgaggggctc aagggcaaga tcagcgaggc ggacaagaag |
| 1921 | aaggttctgg acaagtgtca agaggtcatc tcgtggctgg acgccaacac cttggccgag |
| 1981 | aaggacgagt ttgagcacaa gaggaaggag ctggagcagg tgtgtaaccc catcatcagc |
| 2041 | ggactgtacc agggtgccgg tggtcccggg cctggcggct tcggggctca gggtcccaag |
| 2101 | ggagggtctg ggtcaggccc taccattgag gaggtggatt aggggccttt gttctttagt |
| 2161 | atgtttgtct ttgaggtgga ctgttgggac tcaaggactt tgctgctgtt ttcctatgtc |
| 2221 | atttctgctt cagctctttg ctgcttcact tctttgtaaa gttgtaacct gatggtaatt |
| 2281 | agctggcttc attatttttg tagtacaacc gatatgttca ttagaattct ttgcatttaa |
| 2341 | tgttgatact gtaagggtgt ttcgttccct ttaaatgaat caacactgcc accttctgta |
| 2401 | cgagtttgtt tgtttttttt tttttttttt ttttttgctt ggcgaaaaca ctacaaaggc |
| 2461 | tgggaatgta tgtttttata atttgtttat ttaaatatga aaaataaaat gttaaacttt |
| 2521 | aaaaaaaaaa aaaaaaaaaa aaaaaaaaaa a |

### Examples

Treatment of Mutant VCP Mice and VCP Patient iPSC-Derived Motor Neurons with Arimoclomol Ameliorates FTD and ALS Pathology

### Introduction

Fronto-temporal Dementia (FTD) is the most common type of dementia presenting in those under the age of 65, with an incidence of approximately 3.5 per 100,000 in England while, Amyotrophic lateral sclerosis (ALS) has an incidence of 2 per 100,000. Unfortunately, to date, there is no cure for either of these debilitating diseases. While extensive research effort is directed towards identifying the cause of these diseases, there is clear evidence of protein dyshomeostasis in the brain and spinal cord with the presence of misfolded and aggregated proteins (1). Valosin containing protein (VCP) is a central protein in normal protein degradation pathways. Mutations in this protein can give rise to ubiquitin-positive proteinaceous aggregates and mislocalisation of nuclear TDP-43, an RNA modulating protein which become translocated to the cytoplasm. As these are both prominent pathological features of both FTD and ALS targeting protein mishandling may be an effective therapeutic approach for these diseases.

To investigate this possibility, we studied the effects of augmenting the heat shock response (HSR) in neural tissues of a transgenic mouse model of multisystem proteinopathy, also known as Inclusion Body Myopathy with early-onset Paget disease and frontotemporal dementia (IBMPFD). Pathology in these mice is caused by over-expression of mutant human VCP (A232E mutation) which causes the severest form of multisystem proteinopathy also in patients. In addition, we also examined the effects of arimoclomol in human iPSC-derived motor neurons from mVCP patients and confirmed

The HSR is an endogenous cytoprotective response to cell stress, which involves an upregulation in the expression of key molecular chaperones called heat shock proteins (HSP), in an attempt to improve protein handling and restore cellular protein homeostasis. We have previously shown that pharmacological up-regulation of the heat shock response (HSR), with a co-inducer of the HSR called Arimoclomol, attenuates disease in mouse models of neurodegenerative diseases including ALS (2) as well as Spinal Bulbar Muscular Atrophy (3).

In addition we have recently shown that treatment with Arimoclomol attenuates muscle pathology in mutant VCP (mVCP) mice, which recapitulates characteristic features of the inflammatory myopathy Inclusion body myopathy (IBM) in skeletal muscle (4,5). These results showed that treatment of mVCP mice with Arimoclomol led to decreased protein aggregation and TDP-43 mislocalisation, as well as reduced myofibre atrophy and degeneration. While we observed no significant reduction in grip strength relative to body weight in transgenic mice with wildtype human VCP (wt-VCP) between 4 to 14 months of age, mVCP mice showed a significant, 44.1% reduction in grip strength during this period. Interestingly, in mVCP mice treated with Arimoclomol, there was no significant decline in grip strength throughout the duration of the study. Furthermore, in vivo assessment of maximal tetanic force of the hindlimb extensor digitorum longus (EDL) muscles of 14 month old mice revealed a significant decrease in force generated by EDL muscles of mVCP mice compared to wt-VCP controls. However, treatment of mVCP mice with Arimoclomol prevented this reduction in muscle force. These results show that there is a significant loss of muscle force in mVCP mice between 4 and 14 months of age, and that this is prevented by chronic treatment with Arimoclomol.

These beneficial effects of Arimoclomol on muscle pathology in mVCP mice are likely to result, at least in part, to an increase in the expression of HSPs, since Western blot analysis of muscle from mVCP mice treated with Arimoclomol showed a two-fold increase in the expression of HSP70 compared to that of untreated mVCP mice.

Methods

Transgenic Mouse Colonies: Colonies of mutant Valosin Containing Protein (VCP) (A232E) and control wild type VCP (wtVCP) transgenic mice were maintained at the UCL Institute of Neurology under license from the UK Home office.

Arimoclomol treatment: Male mutant VCP (mVCP) mice were treated with Arimoclomol (120 mg/kg daily; orally in drinking water), from the start of symptom onset at 4 months, until close to end-stage at 14 months. Transgenic mice over-expressing wild-type human VCP (wt-VCP) were used as controls, and 10 male mice per group were studied; a sample size sufficient to test for statistical significance at P<0.05 in a single sex group.

Motor Unit Counts:

In vivo physiology was carried out acutely at 14 months of age on terminally anaesthetized mice to quantify the number of motor units in the extensor digitorum longus (EDL) muscle in the hindlimb of mice in all experimental groups. Briefly, Isometric contractions were elicited by stimulating the Extensor Digitorum Longus (EDL) motor nerve using pulses of 0.02 ms duration and supramaximal intensity via electrodes. Contractions were elicited by stimulation of the sciatic nerve. The number of motor units in the EDL muscles was determined by stimulating the motor nerve with stimuli of increasing intensity, resulting in stepwise increments in twitch tension because of successive recruitment of motor axons.

Motor Neuron Counts and Area Measurements:

20 μm spinal cord sections from L3-L6 were stained with gallocyanin to visualise neurons for quantification (5 mice per group). Sciatic pool motor neurons were counted from every 3rd section as seen under a Leica light microscope. 20 images of the sciatic pool regions of the spinal cord were taken per animal to measure the size distribution of motor neurons present in this area (at ×20 magnification). A minimum of 3 mice per experimental group were assessed. The soma area of motor neurons was determined by drawing around individual Nissl stained (gallocyanin) motor neuron cell bodies in cross-sectional images of L4-L5 region of the spinal cord. This was recorded using Leica Application Suite V3.8 analysis software and presented as a percentage of the total number of motor neurons per group.

Immunohistochemistry:

Brain and spinal cords were harvested from mice in all experimental groups following transcardial perfusion with saline followed by 4% paraformaldehyde (PFA). Brain and spinal cords were then kept in 4% PFA for 12 hours before being transferred to a 30% sucrose solution. Cross-sections of brain and spinal cords were cut at 20 μm and blocked in 10% Normal Goat serum with 0.1% Triton X100 in PBS before primary antibodies were added for 1 hour at room temperature. Primary antibodies: Rabbit anti-TDP-43 [1:500], Rabbit anti-ubiquitin [1:500], mouse anti-phospho tau (AT8) [1:100], Rabbit/mouse anti-Beta-3 tubulin [1:100], mouse anti-HSP70 [1:100], Mouse anti-p62 [1:200], Rabbit anti-LC3 [1:500]Rabbit anti-Iba1 [1:100], anti neurofilament 2H3 [1:20], Synaptic vesicle protein [1:20], Fluoromyelin Red myelin stain [1:300]. Fluorescently labelled or biotinylated secondary antibodies were used 1:1000 for 2 hours at room temperature. 4′6-Diamidino-2-Phenylindole, Dihydrochloride (DAPI) was used to counterstain for nuclei in all fluorescent images. Imaging of tissue was using a standard Leica light/fluorescent microscope or a LSM 780 confocal microscope.

α-Bungarotoxin-tetramethylrhodamine was used to fluorescently label neuromuscular junction endplates for 1 hour at RTP.

Fluorescent images were visualised under a Leica fluorescent microscope and analysed using Leica Application Suite software (Leica Microsystems, Germany).

Human iPSC Derived Motor Neuron Generation:

iPSCs were maintained on Geltrex (Life Technologies) with Essential 8 Medium media (Life Technologies), and passaged using EDTA (Life Technologies, 0.5 mM). All cell cultures were maintained at 37° C. and 5% carbon dioxide. Motor neuron (MN) differentiation was carried out using a previously published protocol (Hall et al., 2017). Briefly, iPSCs were first differentiated to neuroepithelium by plating to 100% confluency in chemically defined medium consisting of DMEM/F12 Glutamax, Neurobasal, LGlutamine, N2 supplement, non-essential amino acids, B27 supplement, 3-mercaptoethanol (all from Life Technologies) and insulin (Sigma). Treatment with small molecules from day 0-7 was as follows: 1 μM Dorsomorphin (Sigma), 2 μM SB431542 (Sigma), and 3 μM CHIR99021 (Sigma). At day 8, the neuroepithelial layer was enzymatically dissociated using dispase (GIBCO, 1 mg/ml), plated onto Geltrex coated plates and next patterned for 7 days with 0.5 μM retinoic acid and 1 μM Purmorphamine. At day 14 spinal cord MN precursors were treated with 0.1 μM Purmorphamine for a further 4 days before being terminally differentiated in 0.1 μM Compound E (Sigma) to promote cell cycle exit. Cells were treated with 10 μM Arimoclomol for 24 hours following terminal differentiation and fixed in PFA for immuno-labelling.

Human Brain Samples:

Frozen human brain samples were obtained from the Queen Square Brain Bank for Neurological Disorders, UCL Institute of Neurology. Cortical sections were received cryosectioned at 12 μm onto glass slides. Immunohistochemistry and immunofluorescent staining was conducting using standard histology protocols. Primary antibodies used are as follows: Rabbit anti-TDP-43 [1:500], mouse anti-HSP70 [1:100], Mouse anti-p62 [1:200], Rabbit anti-LC3 [1:500]. DAPI was used 1:1000 to label nuclei.

### Results

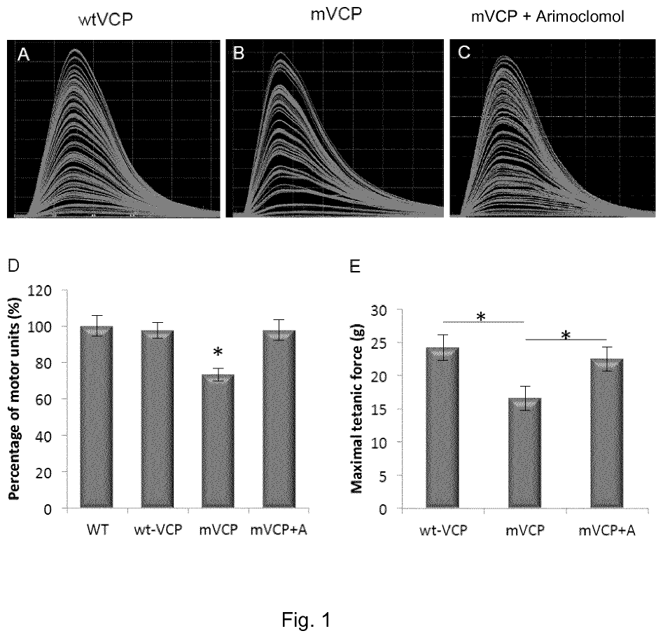

Loss of motor units and neurons in mVCP mice is prevented by Arimoclomol treatment Physiological data from our in vivo study has shown that there is a reduced number of motor units innervating the hindlimb muscles of mVCP mice compared to wild-type VCP controls, and that this reduction in motor unit survival is prevented in mVCP mice treated with Arimoclomol (FIG. 1). These findings suggest there is likely to be significant motor neuron degeneration in mVCP mice that may contribute to the observed muscle weakness detected in mVCP mice.

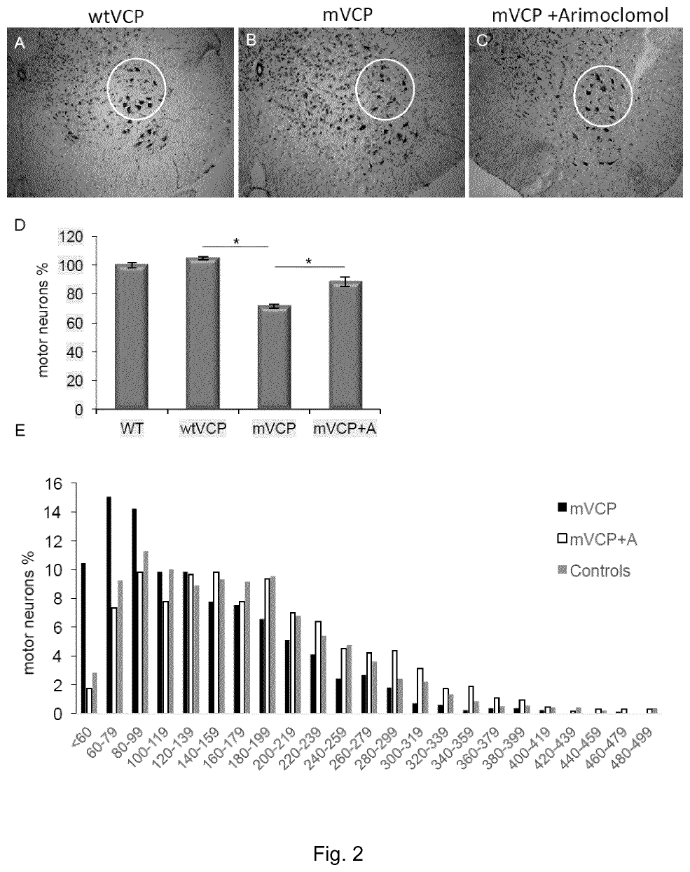

Quantification of the number of motor neurons in the sciatic pool (L3-6) of the spinal cord reveals a significant reduction in motor neuron survival in mVCP mice compared to controls (FIG. 2). This reduction in motor neuron survival is prevented in mice treated with Arimoclomol. Assessment of motor neuron soma size showed a clear shift towards smaller motor neuron size in mVCP mice compared to controls, suggesting a loss of larger (alpha) motor neurons. This shift is not seen in the Arimoclomol treated mice.

TDP-43 Pathology in mVCP Mice is Attenuated with Arimoclomol Treatment

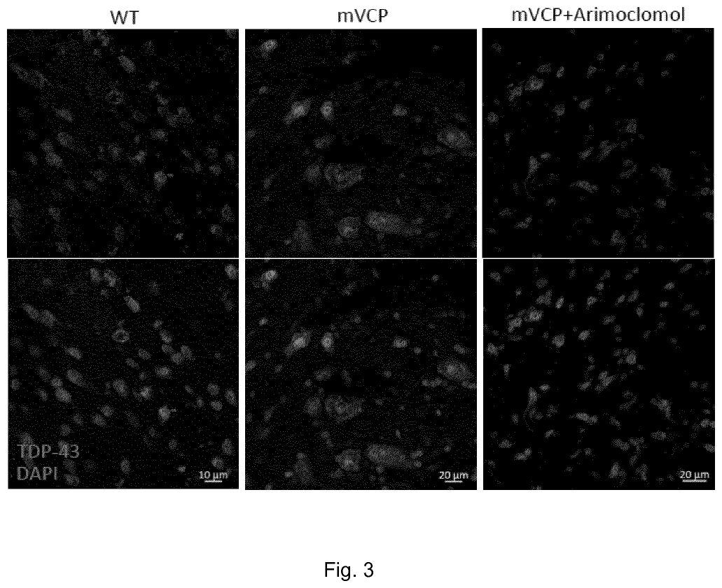

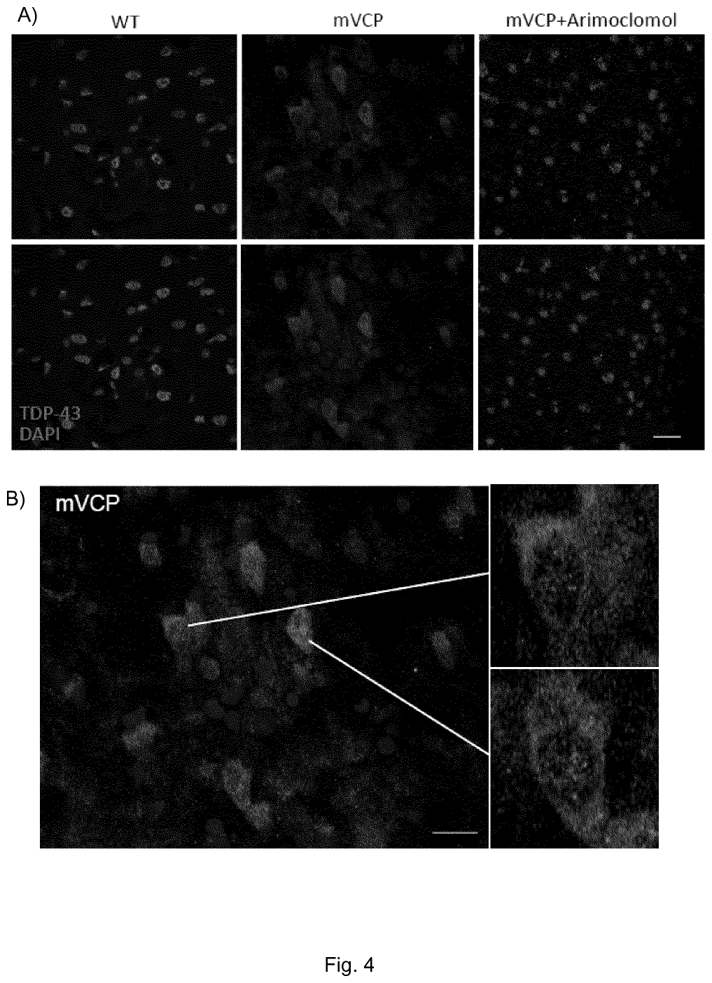

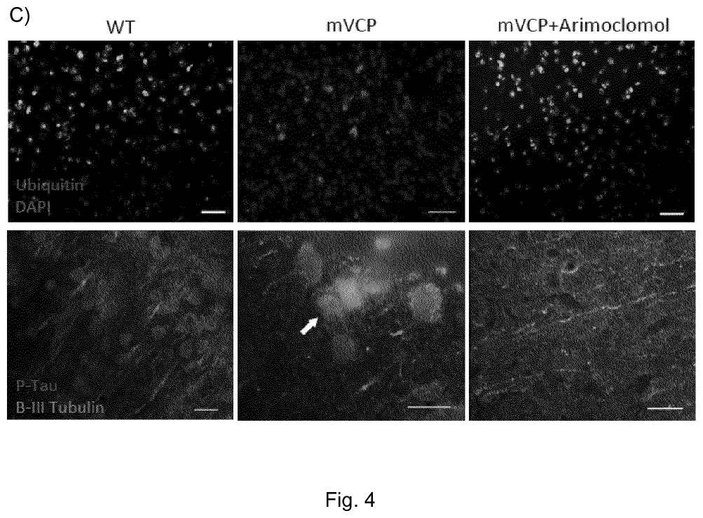

The C-terminal portion of the nuclear protein TDP-43 becomes mislocalised to the cytoplasm in the brain and spinal cord of mVCP mice, with nuclear clearance of TDP-43 observed in brain tissue (FIGS. 3 and 4), a pathological feature in both ALS and FTD patients. TDP-43 mislocalisation was reduced in mVCP mice treated with Arimoclomol. No cytoplasmic TDP-43 immunostaining was observed in wtVCP or non-transgenic controls.

Intracellular Ubiquitin Protein Aggregation and Extracellular p-Tau Detected in m VCP Mice is not Detected in mVCP Mice Treated with Arimoclomol

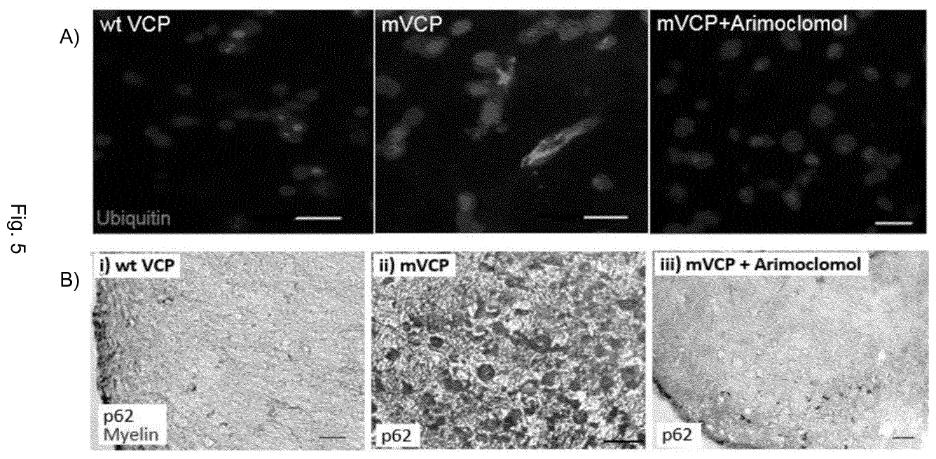

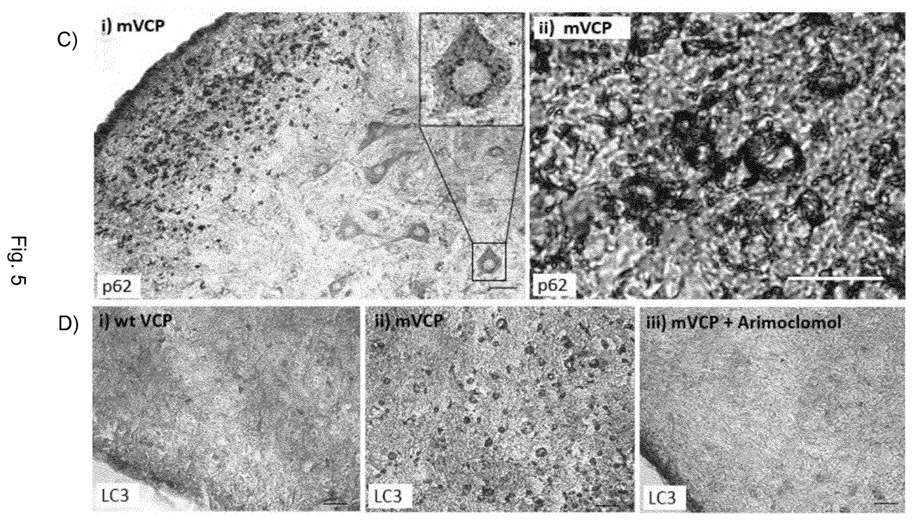

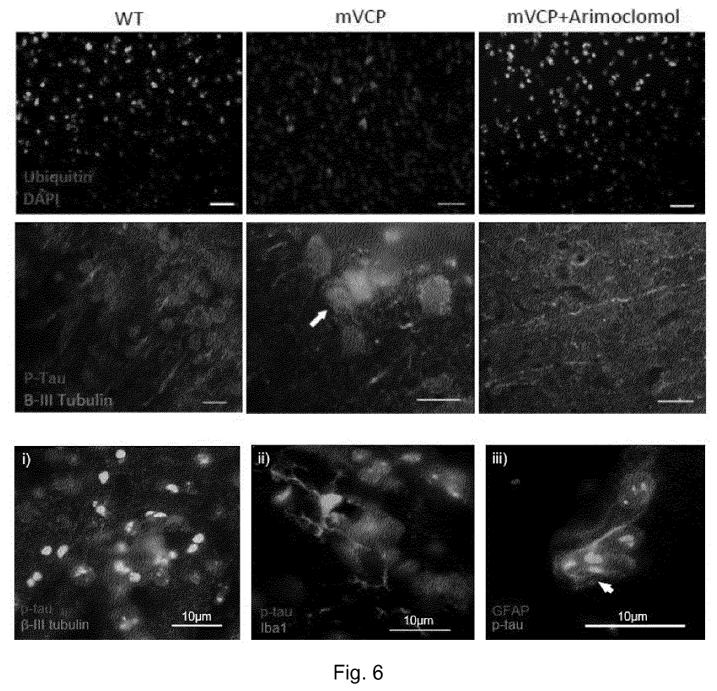

mVCP mice develop ubiquitin-positive intracellular aggregates in both brain and spinal cord tissue (FIGS. 5 and 6). p62 positive aggregates are also observed in spinal cord motor neurons. Phosphorylated tau-positive (p-tau) extracellular aggregates/lesions are present in the brain of mVCP mice which are not observed in wildtype controls (FIG. 6). These lesions are associated with glial cells which are immunoreactive for the microglial marker Iba1 or the astroglial marker GFAP, suggesting an attempt by the brain to ameliorate pathology. Arimoclomol treatment prevents the formation of these proteinaceous aggregates in mVCP mice. No difference in ubiquitin or p-tau immuno-reactivity was observed in wtVCP controls compared to non-transgenic controls.

Increased Protein Degradation in Grey and White Matter and Myelin Degeneration in mVCP Spinal Cord is Improved by Arimoclomol Treatment

p62 (sequestosome) shuttles aberrant proteins to the proteasome and for autophagy for degradation, and LC3 is a marker of autophagy. Our results show a substantial increase in p62 expression in spinal cord white and grey matter in mVCP mice compared to controls (FIGS. 5 B and C). p62 aggregates were observed in motor neurons, and intense p62 staining was observed in oligodendrocytes co-labelled with fluoro-myelin. Higher magnification images of these oligodendrocytes revealed highly disrupted myelination around axons, suggesting axonal or myelin degeneration. This pattern of p62 expression was not seen in controls.

The accumulation of p62, which is normally cleared when associated with proteins undergoing degradation, suggests a possible defect in autophagy in mVCP mice. We therefore looked at the expression of LC3, a protein which is recruited to the autophagosomal membrane before being degraded in the autolysosomal lumen, thereby indicating autophagic activity in a cell (9). In mVCP spinal cord, we detected a substantially increased expression of LC3 in oligodendrocytes associated with abnormal myelin, providing further evidence of defective autophagy in these cells (FIG. 5 D). This pattern of LC3 expression and abnormal myelination was not observed in transgenic control animals. However, accumulation of p62, myelin abnormalities and increased LC3 expression was significantly ameliorated in mVCP mice treated with Arimoclomol.

Arimoclomol Treatment Enhances HSP70 Expression in mVCP Mouse Brain and Spinal Cord

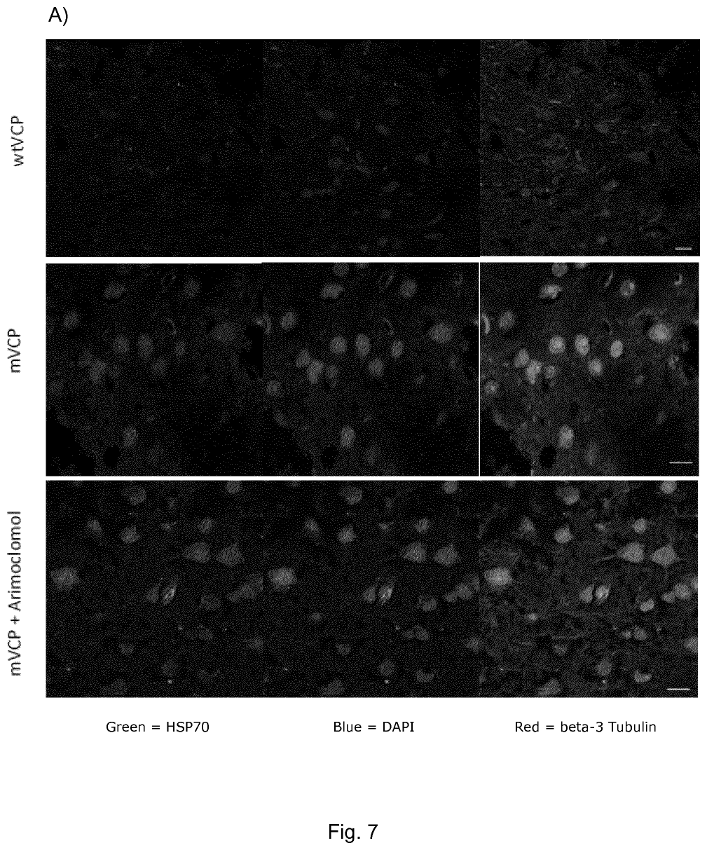

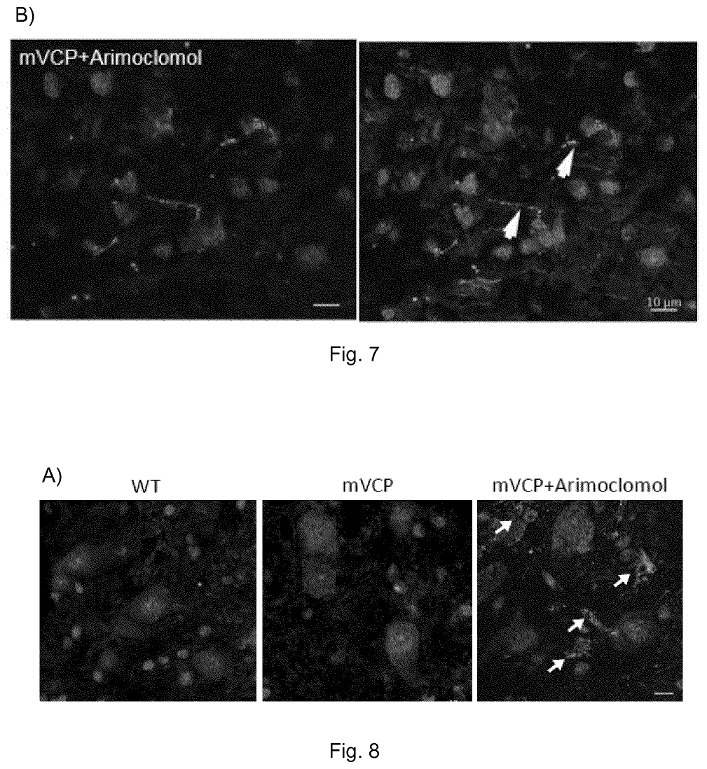

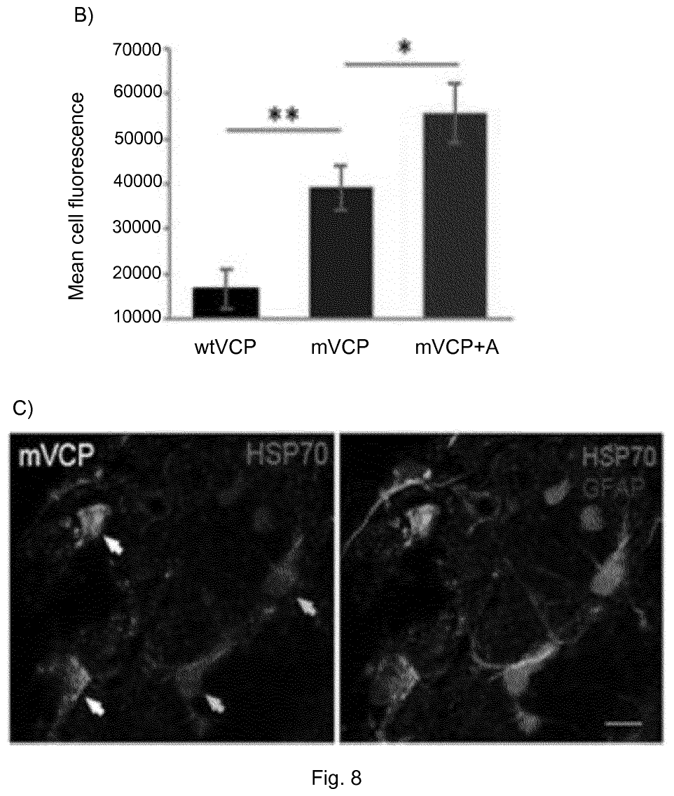

Heat shock protein 70 (HSP70) expression is a key marker of the heat shock response in cells. This protein is increased in the brain and spinal cord of mVCP mice and further augmented in the brain and spinal cord of mVCP mice treated with Arimoclomol (FIGS. 7 and 8) indicating the induction of the heat shock response. Glial cells in the spinal cord and brain in the Arimoclomol-treated mVCP mice also show increased expression of HSP70, suggesting that the neuronal support network may also contribute to neuronal survival through the heat shock response. No difference in HSP70 expression was observed in transgenic and non-transgenic control mice.

Cell Death is Prevented in the Brain of mVCP Mice Treated with Arimoclomol

Mutations in VCP cause <1% of all FTD cases²⁰, and a third of patients diagnosed with multisystem proteinopathy (MSP) caused by mutations in VCP go on to develop FTD⁸. We therefore examined the brain of mVCP mice for FTD-like pathology.

Apoptosis in the brain was assessed by Terminal deoxynucleotidyl transferase (TdT) dUTP Nick-End Labeling (TUNEL) assay for apoptotic cells, where nuclei containing double-stranded breaks in the DNA fluoresce green (Fluoroscein-tagged), indicating DNA degradation at the later stage of apoptosis (FIG. 9). In some mVCP mice, TUNEL-positive nuclei in small areas of layer I in the cortex were detected which were not seen in control animals, indicating brain cells undergoing apoptosis. Treatment of mVCP mice with arimoclomol prevented the appearance of apoptotic cells in the brain of these mice.

Stress Granule Markers Detected in Aggregates in m VCP Cells not Seen in Arimoclomol Treated Animals

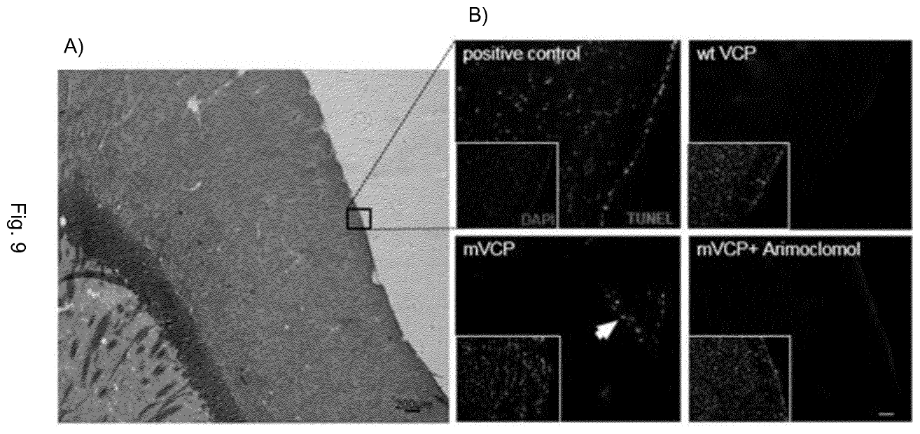

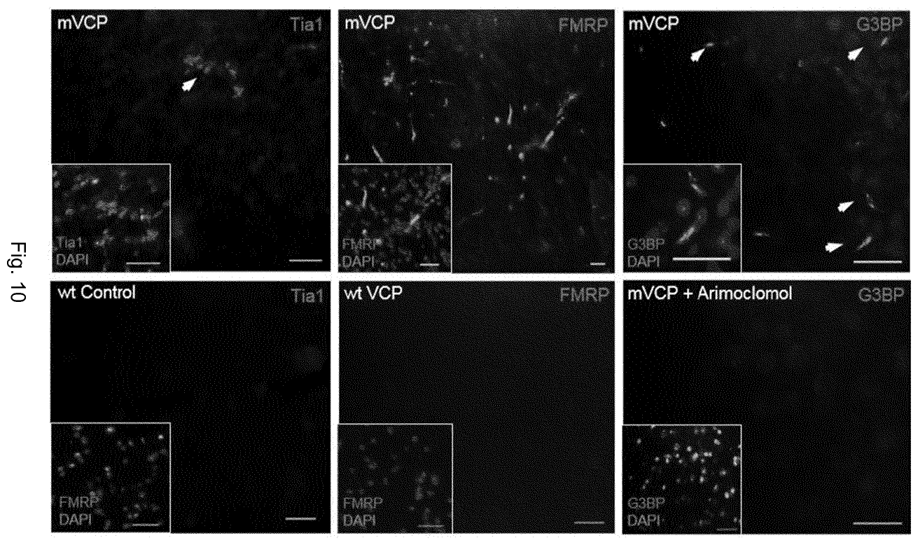

Three markers of stress granules, Tia1, FMRP and G3BP, were used to detect the presence of these RNA-containing structures (FIG. 10). All three markers were found to be abnormally aggregated in the brain of mVCP mice but were not observed in the brain of control animals or in mVCP mice treated with Arimoclomol.

Neuromuscular Junction (NMJ) Defects are Prevented in mVCP Mice Treated with Arimoclomol

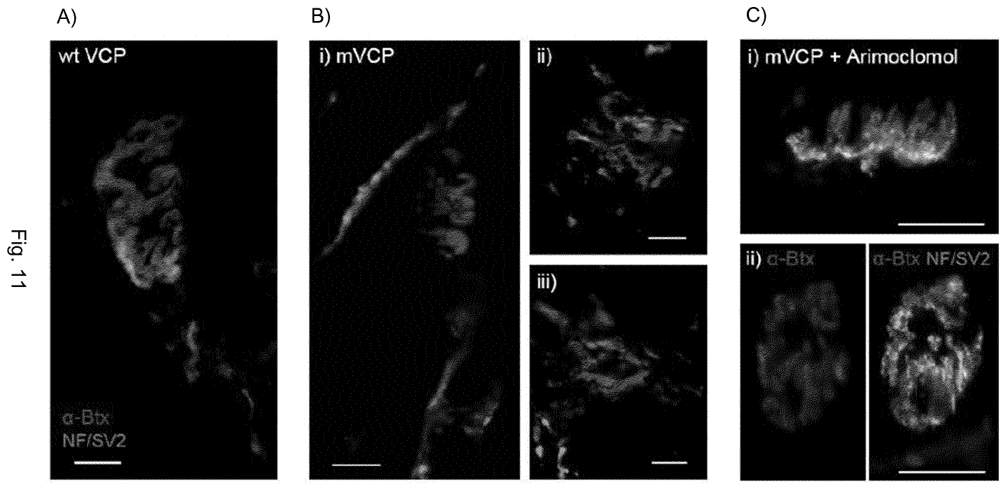

The NMJ is the chemical synapse connecting a motor neuron to the muscle fibre it innervates and therefore preservation of its morphology and function is crucial for muscle contraction to be elicted. In mVCP mice, there was clear evidence of NMJ disruption and denervation (FIG. 11), which corroborates with our findings of muscle function deficits in the same group of mice. These defects of the NMJ and denervation was not observed in muscles of arimoclomol treated mVCP mice These disrupted NMJ structures were not seen in control mice or in mVCP mice treated with Arimoclomol.

Pathological Hallmarks of VCP Pathology are Present in mVCP Patient-Derived iPSC Motor Neurons and are Improved Following Treatment with Arimoclomol.

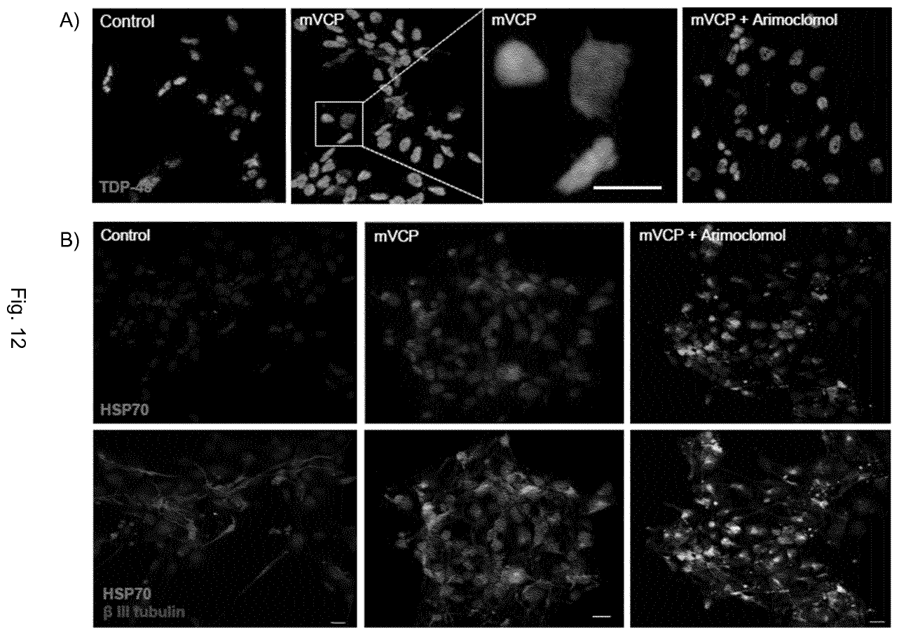

TDP-43 mislocalisation is a characteristic hallmark for both FTD and ALS pathology. In this study we assessed the expression pattern of TDP-43 in iPSC-derived motor neurons derived from patients with mutations in VCP (FIG. 12 A). We detected cytoplasmic mislocalisation of TDP-43 in mVCP iPSC motor neurons which were not seen in control cells. Mislocalised TDP-43 was ameliorated in iPSC-derived motor neurons following treatment with arimoclomol treatment. Importantly, this pathology was associated with an increased level of HSP70 expression (FIG. 11 B) indicating the HSR has been triggered in these cells. Following Arimoclomol treatment, HSP70 expression was substantially increased, suggesting the drug is able to co-induce the HSR, augmenting the presence of HSP70 in these human-derived cells

TDP-43 Mislocalisation and Increased HSP70 Levels are Present in Human FTD Patient Brain Tissue

To confirm that the pathology observed in mVCP mouse brain and spinal cord, and mVCP patient iPSC-derived motor neurons, and that the beneficial effects of arimoclomol on these characteristics are clinically relevant, we also assessed the pattern of TDP-43 expression in port-mortem tissue from patients with different forms of fronto-temporal dementia (FTD; FIG. 12). These patients had different forms of FTD, namely FTD with MND, with ubiquitin-positive inclusion bodies, with TDP-43 mutation or FTD associated with tau pathology. In the cortex tissue of all four patients, we observed frequent cytoplasmic mislocalisation of TDP-43 in brain cells which was rarely seen in brain tissue from aged-matched control individuals. Furthermore, while HSP70 levels in control tissue was only detectable at a low level, in all four patient brain samples, HSP70 expression was notably upregulated suggesting an instigation of the HSR in response to cell stress.

Protein Degradation Markers Seen in mVCP Mice are Also Present in FTD Patient Brain

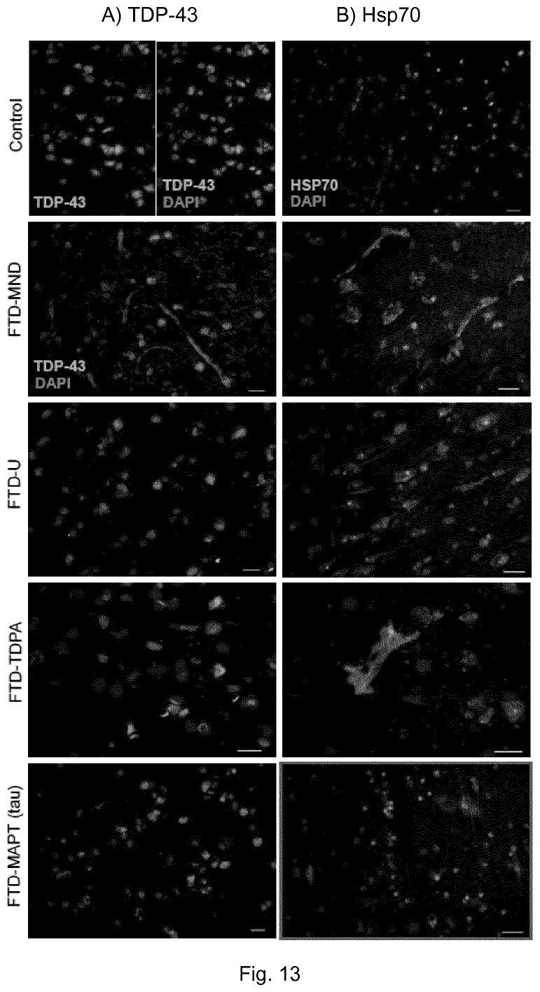

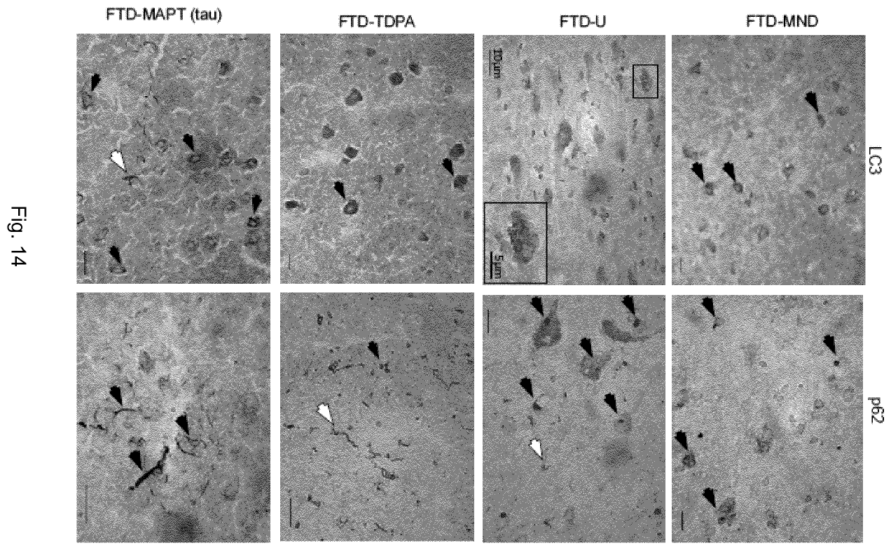

We also assessed the expression of markers of protein degradation, p62 and LC3, in FTD patient brain, which were both altered in brain of mVCP mice. Both p62 and LC3 were present in cytoplasmic aggregates in all four patient samples (FIG. 13). p62 was present in neurites in FTD-U and in FTD-TDPA, and was associated with neurofibrillary tangles in FTD-MAPT (tau). LC3 was also abnormally associated with these structures, suggesting at attempt by the cells to perhaps degrade the misfolded tau protein causing pathology.

### Discussion

We have previously shown that muscle pathology in mVCP mice is attenuated following treatment with Arimoclomol (5). In this study we have extended these findings to examine the effects of Arimoclomol on the brain and spinal cord of these mice with a mutation in the key protein-handling protein VCP and corroborated our findings in VCP patient-derived iPSC motor neurons. Furthermore, the key pathological features of disease observed in mVCP mouse spinal cord and brain which is ameliorated following treatment with arimoclomol, are also a feature of pathology in postmortem human brain tissue from patients with a number of forms of FTD.

In mVCP mice, skeletal muscles recapitulate characteristic features of the inflammatory myopathy Inclusion body myositis including formation of ubiquitinated aggregates, TDP-43 mislocalisation, as well as changes in mitochondrial morphology, function and degeneration of muscle fibres. These myopathic changes correlated with a reduction in grip strength in mVCP mice compared to controls. Treatment with Arimoclomol attenuated all of these disease features in mVCP mice (5).

Electrophysiological assessment of the mVCP mice has shown that the decline in muscle strength and muscle force generation corresponds to a reduction in the number of motor units. In these studies, the hind limb muscle EDL was assessed in the mVCP mice for maximal tetanic force generation which revealed a 31.5% reduction in force (5), correlating with a 30% reduction in the number of EDL motor units (FIG. 1). In line with this, the number of surviving motor neurons in the sciatic pool of the mVCP mice, which innervate the hindlimb muscles, was reduced by approximately 30% (FIG. 2).

Two motor neuron sub-types are present on the spinal cord motor pool—large alpha neurons, which innervate extrafusal muscle fibers of skeletal muscle and are directly responsible for initiating their contraction, and smaller gamma neurons which innervate the intrafusal muscle fibres of muscle spindles, specialized sensory organs. Alpha motor neurons are selectively vulnerable in ALS. Examination of the size distribution of sciatic motor neurons show a clear shift in the size distribution of surviving motor neurons on mVCP mice, towards smaller neurons compared to WT and wtVCP controls (FIG. 2). This finding indicates that it is the larger alpha motor neurons that degenerate in mVCP mice, as has been reported in the SOD1^{G93}A mouse model of ALS (2).

In mVCP mice treated with Arimoclomol from the start of symptom onset at 4 months of age to 14 months of age, motor neuron survival is improved and motor unit number is maintained. There is also no significant change in the size distribution of motor neurons in mVCP mice treated with Arimoclomol compared to controls, with little or no shift in size distribution compared to controls. As a result, the muscle force generated by the EDL muscle in Arimoclomol treated mVCP mice is significantly greater than in untreated mice (FIG. 1).

To investigate the pathological changes that may have played a role in the death of motor neurons in mVCP mice and reduction of muscle function, key pathological changes which are hallmarks of neurodegenerative diseases were investigated in these cohorts of mice.

TDP-43 (transactive response DNA binding protein 43 kDa) is a protein involved in RNA metabolism and is ubiquitously expressed in most tissues, normally within the nucleus of cells (6). This RNA-binding protein is cleaved by activated Caspase 3/7 following cell stress cytoplasm (7). The translocated C-terminus of TDP-43 is detected in the brain and spinal cord of ALS and FTD patients. In mVCP mice, we observed an increase in mislocalised TDP-43 in the brain and spinal cord compared to control mice. However, in mice treated with Arimoclomol there was a clear reduction in cytoplasmic staining for TDP-43 (FIGS. 3 and 4). Nuclear clearance of TDP-43 was also observed in brain cells, thought to be a pathogenic process which precedes inclusion body formation (10) and links protein aggregation to TDP-43 mislocalisation.

Protein dyshomeostasis has been proposed to play a key role in the pathogenesis of neurodegenerative diseases in which protein aggregation is commonly observed (1). Analysis of protein aggregation in the mVCP mice showed cytoplasmic ubiquitin-positive aggregates in the spinal cord and brain with aggregates seen in the cortex and midbrain (FIGS. 5 and 6). In mVCP mice treated with Arimoclomol, these aggregates were not detected. Mutations in VCP have been shown to impair autophagy, a key protein degradation process essential for maintaining the proteostasis and therefore preventing the aggregation of aberrant proteins in the cell (11). Mutations in VCP have been identified to prevent maturation of autophagosomes, thereby leading to an accumulation of undegraded proteins. In this study we show that a key protein associated with autophagosome function called LC3 is accumulated in oligodendrocytes (FIG. 5 D). Oligodendrocytes are responsible for myelination of spinal cord axons around which they wrap in a typical ‘onion-bulb’ structure seen in cross-sections, and axonal degeneration can result from disrupted myelination (12). However, in our study oligodendrocytes are seen in the mVCP mice to have structural abnormality likely to be a result of defective autophagy. p62 is a protein which shuttles abnormal proteins for degradation by proteasomal degradation and via autophagy. In the mVCP mice p62 is also seen to be increasingly expressed in neurons as aggregates and in myelin-labelled oligodendrocytes in the spinal cord, supporting the picture of defective autophagy in these animals and protein aggregation (FIGS. 5 B and C). Arimoclomol treatment ameliorated these pathological features in the mVCP mice and indicates that upregulating the HSR is beneficial, possibly by reducing the abnormal protein load in the cell through chaperoning activity.

FTD is commonly referred to as a tauopathy due to the presence of hyper-phosphorylated tau (p-tau) lesions in the brains of FTD patients (8). Interestingly, in the brain of mVCP mice, immunostaining for phosphorylated tau (antibody AT8) revealed large extracellular lesions in the cortex, which were not present in the control animals (FIG. 6). These lesions were seen to be associated with glial cells such as Iba1-positive microglia which are part of the brain's rapid response to local injury (13). In mVCP mice that were treated with Arimoclomol, no p-tau lesions were detected, indicating an improvement in this key hallmark of dementia.

Interestingly, cell death was noted in the brain of mVCP mice following a TUNEL apoptosis assay which revealed dying cells in layer 1 of the cortex (FIG. 9), further indicating the stress caused by the pathological changes in the brain. Apoptotic cells were not detected in control and Arimoclomol treated animals. To assess cell stress, we looked for markers of stress granules in the brain of the mice. Stress granules are relatively transient complexes of RNA-binding proteins and key RNA molecules which are sequestered by the cell during stress (14). It is suggested that stress granule assembly and disassembly are regulated by autophagy and persistent stress granules in the cytoplasm may give rise to aggregates. Disruption in the autophagic pathway may therefore contribute to stress granules persisting in the cell and lead to protein aggregation. In our study we used a panel of stress-granule markers to study these complexes in the brain and detected aggregates containing all 3 markers, Tia1, G3BP and FMRP, in the mVCP mice which were not seen in control animals or those treated with Arimoclomol (FIG. 10). This result supports the indication that mutant VCP leads to disruptive autophagy, which in turn affects RNA and protein homeostasis leading to pathological changes such as protein aggregation. Indeed, TDP-43, an RNA-binding protein itself which is mislocalised in the mVCP mice, is a known component of stress granules in the cytoplasm and is therefore part of the cell's stress response (14).

To determine whether the improvements in brain and spinal cord pathology observed in Arimoclomol treated mVCP mice were a result of the co-induction of the HSR, these tissues were immuno-stained for HSP70. HSP70 expression was upregulated in the brain and spinal cord of mVCP mice compared to control animals (FIGS. 7 and 8). However, in mVCP mice treated with Arimoclomol, the expression of HSP70 was further augmented, indicating amplification of the HSR and corroborating the data from our study in the muscle of this mouse model. Interestingly, in both the spinal cord and brain of Arimoclomol-treated mVCP mice, HSP70 was upregulated in glial cells as well as neurons.

Our results show clear signs of neuronal death in the brain and spinal cord of mVCP mice, reminiscent of human FTD and ALS, and link this degeneration to our previously published data which also shows pathology in the muscle of mVCP mice. To determine whether pathology seen in spinal cord motor neurons affects the interface with muscle fibres at the neuromuscular junction we examined the neuromuscular junction. Our results show evidence for disrupted endplate structure and denervation in muscle sections of mVCP mice (FIG. 11) which was not seen in control animals or in mVCP mice treated with Arimoclomol.

To test whether the data from our in vivo mVCP mouse studies was corroborated in human cells, we examined mVCP-patient derived iPSC motor neurons. We focused on TDP-43 cytoplasmic mislocalisation as a key pathological outcome measure in ALS and FTD, and a pathological feature of all three tissues assessed in vivo in mVCP mice (ie in muscle, in spinal cord and in the cortex). Under basal conditions mVCP patient iPSC-motor neurons showed cytoplasmic mislocalisation of TDP-43, which was not observed in cells from healthy controls or importantly, in cells treated with Arimoclomol. Moreover, HSP70 levels in mVCP MNs was increased under basal conditions compared to healthy controls, and was augmented in mVCP patient iPSC-motor neurons treated with Arimoclomol, demonstrating successful co-induction of the HSR by arimoclomol in human neurons.

In order to confirm that the key pathological features observed in tissues of mVCP mice and in mVCP patient iPSC-derived motor neurons which are ameliorated by treatment with arimoclomol are a good readout of the human disease, we also examined the expression of TDP-43 and HSP70 in postmortem samples of brain from patients with a range of FTD subtypes, including a patient with FTD-MND. In all patient brains we identified cells containing mislocalised TDP-43 and an upregulation of HSP70 levels. In addition we also examined signs of disrupted autophagy and protein mishandling in the FTD patient brain samples and found evidence of cytoplasmic LC3 and p62 aggregates in neurons and glia, with p62 also associating with neurofibrillary tangles in the brain of an FTD-MAPT patient. These findings indicate that common pathomechanisms may be the cause of disease in all these patients.

In conclusion, the results of this study show that expression of mutant VCP in transgenic mice results in brain and spinal cord pathology reminiscent of FTD and ALS respectively, replicating key pathological hallmarks of these neurodegenerative diseases, including TDP-43 mislocalisation, ubiquitin-positive and p62 positive protein aggregation as well as lesions of phosphorylated tau in the brain and cell death in both the spinal cord and brain. Denervation at the NMJ and abnormal endplate structure links the neuronal findings to the myopathy seen previously and demonstrates that multiple tissues can be affected in mVCP mice, as observed in patients with multisystem proteinopathy (MSP) where FTD, ALS and IBM can all coexist in individual patients. Importantly, in this study we have shown that in both the mouse model and the patient-derived MNs, treatment with Arimoclomol led to an amelioration of all the pathological changes observed. Since the same pathological characterizes are also observed in FTD patient postmortem brain tissue, these results suggest that induction of Hsp70 exemplified by treatment with Arimoclomol in FTD patients may be a beneficial therapeutic strategy.

### REFERENCES

1. Gotzl J K, et al. (2016) Aging Res Rev. December; 32:122-139
2. Kieran D, Kalmar B, Dick J R et al. (2004) Nat Med; 10:402-405
3. Malik B et al, (2013) Brain 136:926-43
4. Custer S K, Taylor J P et al. (2010) Hum Mol Genet. 1; 19(9):1741-55
5. Ahmed M et al. (2016) Sci. Tran Med; March Vol 8 Issue 331
6. Ratti A, Buratti E, (2016) J Neurochem August; 138 Suppl 1:95-111
7. Quan L et al (2015) Nat Comms 6; 6183
8. Yoshiyama Y et al (2001) Curr Neurol Neurosci Rep. September; 1(5):413-21
9. Tanida, I., Ueno, T. & Kominami, E. LC3 and Autophagy. Methods Mol Biol 445, 77-88 (2008).
10. Edward B. Lee, Virginia M.-Y. Lee, and J Q. Trojanowski. Nat Rev Neurosci, November 30; 13(1): 38-50 (2011)
11. Tresse, E., et al. Autophagy 6, 217-227 (2010).
12. Makoto Higuchi et al. J Neurosci. October 2005, 25 (41) 9434-9443;
13. Hey-Kyeong Jeong et al Exp Neurobiol. 2013 June; 22(2): 59-67.
14. Monahan Z, Shewmaker F, and Pandey U B, Brain Res. October 15; 1649(Pt B): 189-200 (2016)
15. Ito, Hatano, Suzuki (2017) Sci Transl Med. Vol. 9, Issue 415, eeah5436
16. Alberti et al. (2017) Front. Mol. Neurosci. Vol. 10:84

1. A bioactive agent that increases the intracellular concentration and/or activity of one or more heat shock proteins for use in the treatment of a frontotemporal disorder.

2. The bioactive agent for use according to claim 1, wherein said frontotemporal disorder is frontotemporal lobar degeneration (FTLD).

3. The bioactive agent for use according to claim 2, wherein said frontotemporal lobar degeneration is FTLD-TDP.

4. The bioactive agent for use according to any one of the preceding claims, wherein said frontotemporal disorder is frontotemporal dementia (FTD).

5. The bioactive agent for use according to any one of the preceding claims, wherein said frontotemporal dementia (FTD) is selected from the group consisting of behavioral variant FTD (FTD), Pick disease (PiD), frontotemporal Dementia (FTD) associated with motor neuron disease (FTD-MND, frontotemporal Dementia (FTD) associated with ubiquitin-positive inclusions (FTD-U), frontotemporal Dementia (FTD) associated with mutant TDP-43 (FTD-TDPA) and ontotemporal Dementia (FTD) associated with tau-positive inclusions (FTD-tau).

6. The bioactive agent for use according to any one of the preceding claims, wherein said frontotemporal disorder is associated with a mutation in the VCP gene.

7. The bioactive agent for use according to any one of the preceding claims, wherein said frontotemporal disorder is associated with a mutation in the VCP gene selected from the group consisting of R93C, R95G, R95C, R95H, I126F, P137L, R155S, R155C, R155H, R155P, R155L, G157R, R159C, R159H, R159G, R191Q, L198W, A232E, T262A, N387H, A439P, A439S and D592N.

8. The bioactive agent for use according to any one of the preceding claims, wherein said frontotemporal disorder is associated with one or more of TDP-43 mislocalisation, cytoplasmic ubiquitin aggregation, motor unit loss, p-tau lesions and p62 and/or LC3 expression or cytoplasmic aggregation.

9. The bioactive agent for use according to any one of the preceding claims, wherein said frontotemporal disorder is associated with stress granule formation.

10. The bioactive agent for use according to any one of the preceding claims, wherein said frontotemporal disorder is inclusion body myopathy (IBM) with early-onset PDB (Paget's disease of bone) and frontotemporal dementia (FTD); IBMPFD.

11. The bioactive agent for use according to any one of the preceding claims, wherein said frontotemporal disorder is inclusion body myopathy (IBM) with frontotemporal dementia (FTD).

12. The bioactive agent for use according to any one of the preceding claims, wherein said frontotemporal disorder is Paget's disease of bone (PDB) with frontotemporal dementia (FTD).

13. The bioactive agent for use according to any one of the preceding claims, wherein said frontotemporal disorder is IBMPFD with amyotrophic lateral sclerosis (ALS) (IBMPFD-ALS).

14. The bioactive agent for use according to any one of the preceding claims, wherein said frontotemporal disorder is selected from the group consisting of frontotemporal dementia (FTD) with amyotrophic lateral sclerosis (ALS) (ALS-FTD), sporadic ALS-FTD, familial ALS-FTD, and familial ALS associated with mVCP (VCP-fALS).

15. The bioactive agent for use according to any one of the preceding claims, wherein said bioactive agent reduces one or more of cytoplasmic ubiquitin aggregation, TDP-43 mislocalisation, motor unit loss, p-tau lesions and p62 and/or LC3 expression or cytoplasmic aggregation and stress granule formation.

16. The bioactive agent for use according to any of the preceding claims, wherein said bioactive agent increases the intracellular concentration and/or activity of one or more heat shock proteins, including Hsp70.

17. The bioactive agent for use according to any of the preceding claims, wherein said bioactive agent is an inducer of Hsp70.

18. The bioactive agent for use according to any of the preceding claims, wherein said bioactive agent is capable of increasing the intracellular concentration of Hsp70 by amplifying Hsp70 gene expression.

19. The bioactive agent for use according to any of the preceding claims, wherein said bioactive agent is capable of increasing the intracellular concentration of Hsp70 by amplifying Hsp70 gene expression, wherein said bioactive agent is a hydroxylamine derivative.

20. The bioactive agent for use according to any of the preceding claims, wherein said bioactive agent is a small molecule inducer of heat shock proteins, including Hsp70, such as a small molecule inducer of Hsp70.

21. The bioactive agent for use according to any of the preceding claims which is selected from N-[2-hydroxy-3-(1-piperidinyl)-propoxy]-pyridine-1-oxide-3-carboximidoyl chloride (arimoclomol), its stereoisomers and the acid addition salts thereof.

22. The bioactive agent for use according to any of the preceding claims, which is selected from the group consisting of
a. the racemate of N-[2-hydroxy-3-(1-piperidinyl)-propoxy]-pyridine-1-oxide-3-carboximidoyl chloride,
b. an optically active stereoisomer of N-[2-hydroxy-3-(1-piperidinyl)-propoxy]-pyridine-1-oxide-3-carboximidoyl chloride,
c. an enantiomer of N-[2-hydroxy-3-(1-piperidinyl)-propoxy]-pyridine-1-oxide-3-carboximidoyl chloride,
d. (+)-R—N-[2-hydroxy-3-(1-piperidinyl)-propoxy]-pyridine-1-oxide-3-carboximidoyl chloride and (−)-(S)—N-[2-hydroxy-3-(1-piperidinyl)-propoxy]-pyridine-1-oxide-3-carboximidoyl chloride,
e. an acid addition salt of N-[2-hydroxy-3-(1-piperidinyl)-propoxy]-pyridine-1-oxide-3-carboximidoyl chloride,
f. N-[2-hydroxy-3-(1-piperidinyl)-propoxy]-pyridine-1-oxide-3-carboximidoyl chloride citrate (BRX-345), and N-[2-hydroxy-3-(1-piperidinyl)-propoxy]-pyridine-1-oxide-3-carboximidoyl chloride maleate, and
g. (+)-R—N-[2-hydroxy-3-(1-piperidinyl)-propoxy]-pyridine-1-oxide-3-carboximidoyl chloride citrate; (−)-S—N-[2-hydroxy-3-(1-piperidinyl)-propoxy]-pyridine-1-oxide-3-carboximidoyl chloride citrate; (+)-R—N-[2-hydroxy-3-(1-piperidinyl)-propoxy]-pyridine-1-oxide-3-carboximidoyl chloride maleate; and (−)-S—N-[2-hydroxy-3-(1-piperidinyl)-propoxy]-pyridine-1-oxide-3-carboximidoyl chloride maleate.

23. The bioactive agent for use according to any of the preceding claims, which is N-[2-hydroxy-3-(1-piperidinyl)propoxy]-3-pyridinecarboximidamide, dihydrochloride (BGP-15), its stereoisomers and the acid addition salts thereof.

24. The bioactive agent for use according to any of the preceding claims, which is selected from 5,6-dihydro-5-(1-piperidinyl)methyl-3-(3-pyridyl)-4H-1,2,4-oxadiazine (iroxanadine), its stereoisomers and the acid addition salts thereof.

25. The bioactive agent for use according to any of the preceding claims, which is selected from the group consisting of
a. the racemate of 5,6-dihydro-5-(1-piperidinyl)methyl-3-(3-pyridyl)-4H-1,2,4-oxadiazine,
b. an optically active stereoisomer of 5,6-dihydro-5-(1-piperidinyl)methyl-3-(3-pyridyl)-4H-1,2,4-oxadiazine,
c. an enantiomer of 5,6-dihydro-5-(1-piperidinyl)methyl-3-(3-pyridyl)-4H-1,2,4-oxadiazine,
d. (+)-5,6-dihydro-5-(1-piperidinyl)methyl-3-(3-pyridyl)-4H-1,2,4-oxadiazine and (−)-5,6-dihydro-5-(1-piperidinyl)methyl-3-(3-pyridyl)-4H-1,2,4-oxadiazine,
e. an acid addition salt of 5,6-dihydro-5-(1-piperidinyl)methyl-3-(3-pyridyl)-4H-1,2,4-oxadiazine,
f. 5,6-dihydro-5-(1-piperidinyl)methyl-3-(3-pyridyl)-4H-1,2,4-oxadiazine citrate, and 5,6-dihydro-5-(1-piperidinyl)methyl-3-(3-pyridyl)-4H-1,2,4-oxadiazine maleate, and
g. (+)-5,6-dihydro-5-(1-piperidinyl)methyl-3-(3-pyridyl)-4H-1,2,4-oxadiazine citrate; (−)-5,6-dihydro-5-(1-piperidinyl)methyl-3-(3-pyridyl)-4H-1,2,4-oxadiazine citrate; (+)-5,6-dihydro-5-(1-piperidinyl)methyl-3-(3-pyridyl)-4H-1,2,4-oxadiazine maleate; and (−)-5,6-dihydro-5-(1-piperidinyl)-methyl-3-(3-pyridyl)-4H-1,2,4-oxadiazine maleate.

26. The bioactive agent for use according to any of the preceding claims, which is selected from N-[2-hydroxy-3-(1-piperidinyl)-propoxy]-3-pyridinecarboximidoyl chloride (bimoclomol) its stereoisomers and the acid addition salts thereof.

27. The bioactive agent for use according to any of the preceding claims, which is selected from the group consisting of
a. the racemate of N-[2-hydroxy-3-(1-piperidinyl)-propoxy]-3-pyridinecarboximidoyl chloride,
b. an optically active stereoisomer of N-[2-hydroxy-3-(1-piperidinyl)-propoxy]-3-pyridinecarboximidoyl chloride,
c. an enantiomer of N-[2-hydroxy-3-(1-piperidinyl)-propoxy]-3-pyridinecarboximidoyl chloride,
d. (+)-R—N-[2-hydroxy-3-(1-piperidinyl)-propoxy]-3-pyridinecarboximidoyl chloride and (−)-(S)—N-[2-hydroxy-3-(1-piperidinyl)-propoxy]-3-pyridinecarboximidoyl chloride,
e. an acid addition salt of N-[2-hydroxy-3-(1-piperidinyl)-propoxy]-3-pyridinecarboximidoyl chloride,
f. N-[2-hydroxy-3-(1-piperidinyl)-propoxy]-3-pyridinecarboximidoyl chloride citrate, and N-[2-hydroxy-3-(1-piperidinyl)-propoxy]-3-pyridinecarboximidoyl chloride maleate, and
g. (+)-R—N-[2-hydroxy-3-(1-piperidinyl)-propoxy]-3-pyridinecarboximidoyl chloride citrate; (−)-S—N-[2-hydroxy-3-(1-piperidinyl)-propoxy]-3-pyridinecarboximidoyl chloride citrate; (+)-R—N-[2-hydroxy-3-(1-piperidinyl)-propoxy]-3-pyridinecarboximidoyl chloride maleate; and (−)-S—N-[2-hydroxy-3-(1-piperidinyl)-propoxy]-3-pyridinecarboximidoyl chloride maleate.

28. The bioactive agent for use according to any of the preceding claims, which is selected from the group consisting of: membrane-interactive compounds such as alkyllysophospholipid edelfosine (ET-18-OCH3 or 1-octadecyl-2-methyl-rac-glycero-3-phosphocholine); anti-inflammatory drugs including cyclooxygenase 1/2 inhibitors such as celecoxib and rofecoxib, as well as NSAIDs such as acetyl-salicylic acid, sodium salicylate and indomethacin; dexamethasone; prostaglandins PGA1, PGj2 and 2-cyclopentene-1-one; peroxidase proliferator-activated receptor-gamma agonists; tubulin-interacting anticancer agents including vincristine and paclitaxel; the insulin sensitizer pioglitazone; anti-neoplastic agents such as carboplatin, doxorubicin, fludarabine, ifosfamide and cytarabine; Hsp90 inhibitors including geldanamycin, 17-AAG, 17-DMAG, radicicol, herbimycin-A and arachidonic acid; proteasome inhibitors such as MG132, lactacystin, Bortezomib, Carfilzomib and Oprozomib; serine protease inhibitors such as DCIC, TLCK and TPCK; Histone Deacetylase Inhibitors (HDACi) including SAHA/vorinostat, Belinostat/PXD101, LB-205, LBH589 (panobinostat), FK-228, CI-994, trichostatin A (TSA) and PCI-34051; anti-ulcer drugs including geranylgeranylacetone (GGA), rebamipide, carbenoxolone and polaprezinc (zinc L-carnosine); heavy metals (zinc and tin); cocaine; nicotine; alcohol; alpha-adrenergic agonists; cyclopentenone prostanoids; L-type Ca++ channel blockers, such as L-type Ca++ channel blockers that also inhibits ryanodine receptors, such as lacidipine; ryanodine receptor antagonists such as DHBP (1,1′-diheptyl-4,4′-bipyridium; as well as herbal medicines including paeoniflorin, glycyrrhizin, celastrol, dihydrocelastrol, dihydrocelastrol diacetate, curcumin, sub-lethal heat therapy and a membrane fluidizer including benzyl alcohol, heptanol, AL721, docosahexaenoic acid, aliphatic alcohols, oleyl alcohol, dimethylaminoethanol, A₂C, farnesol and anaesthetics such as lidocaine, ropivacaine, bupivacaine and mepivacaine.

29. The bioactive agent for use according to any of the preceding claims, which is Hsp70 protein, or a functional fragment or variant thereof.

30. The bioactive agent for use according to claim 28, wherein said Hsp70 is selected from HSPA1A (SEQ ID NO:1 and SEQ ID NO:2) and HSPA1B (SEQ ID NO:4 and SEQ ID NO:5), recombinant Hsp70 (rHsp70), or a functional fragment or functional variant thereof, including a naturally occurring variant of Hsp70, or a fragment of a naturally occurring variant of Hsp70.

31. The bioactive agent for use according to claim 29, wherein said functional fragment or variant of Hsp70 retains the capability of one or more of:
i. reducing cytoplasmic ubiquitin aggregation,
ii. reducing TDP-43 mislocalisation,
iii. reducing motor unit loss
iv. reducing stress granule formation, such as reducing stress granule markers including Tia1, FMRP and G3BP,
v. reducing p-tau positive lesions, and
vi. reducing P62 and/or LC3 expression or cytoplasmic aggregation.

32. A composition, such as a pharmaceutical composition, comprising a bioactive agent that increases the intracellular concentration and/or activity of one or more heat shock proteins, including Hsp70, and optionally one or more pharmaceutically acceptable carriers, for use in the treatment of a frontotemporal disorder.

33. A composition, such as a pharmaceutical composition, comprising—separately or together—a bioactive agent that increases the intracellular concentration and/or activity of one or more heat shock proteins, including Hsp70; and one or more further active ingredients; for use in the treatment of a frontotemporal disorder.
